(19) United States
(12) Patent Application Publication
(10) Pub. No.: US 2004/0047855 A1
(43) Pub. Date: Mar. 11, 2004

(54) Compositions and methods for reducing lung volume

(75) Inventor: Edward Ingenito, Kingston, MA (US)

Correspondence Address: FISH & RICHARDSON PC, 225 FRANKLIN ST, BOSTON, MA, 02110, US

(73) Assignee: Bistech, Inc., a Delaware corporation

(21) Appl. No.: 10/464,115

(22) Filed: Jun. 17, 2003

Related U.S. Application Data
(60) Provisional application No. 60/389,731, filed on Jun. 17, 2002.

(51) Int. Cl. A61K 38/48

(52) U.S. Cl. 424/94.63

(57) ABSTRACT

The invention includes methods for performing non-surgical lung volume reduction in a patient by (a) administering, through the patient's trachea, a composition comprising an enzyme (e.g., a protease, such as a serine protease (e.g., trypsin, chymotrypsin, elastase, or an MMP); and (b) collapsing a region of the lung, at least a portion of which was contacted by the composition administered in step (a). The patient can have COPD (e.g., emphysema) or their lung can be damaged by a traumatic event. The tissue in the targeted area can also include an abscess or fistula. One can similarly treat other tissues (i.e., non-lung tissues) by exposing those tissues to an enzyme-containing composition (or other composition described herein). These tissues may be those that are obscured from a therapeutic agent by epithelial cells or that will contact an implantable device.

This application claims the benefit of the priority date of U.S. Ser. No. 60/389,731, filed Jun. 17, 2002, the entire content of which is hereby incorporated by reference.

### TECHNICAL FIELD

This invention features compositions and methods for treating patients who have certain lung diseases, such as emphysema.

### BACKGROUND

Emphysema, together with asthma and chronic bronchitis, represent a disease complex known as chronic obstructive pulmonary disease (COPD). These three diseases are related in that they each cause difficulty breathing and, in most instances, progress over time. There are substantial differences, however, in their etiology, pathology, and prognosis. For example, while asthma and chronic bronchitis are diseases of the airways, emphysema is associated with irreversible, destructive changes in lung parenchyma distal to the terminal bronchioles. Cigarette smoking is the primary cause of emphysema; the smoke triggers an inflammatory response within the lung, which is associated with an activation of both elastase and matrix metallo-proteinases (MMPs). These enzymes degrade key proteins that make up the tissue network of the lungs (Shapiro et al., Am. J. Resp. Crit. Care Med. 160:s29-s32, 1999; Hautamaki et al., Science 277:2002-2004, 1997). In fact, the pathological determinant of lung dysfunction in emphysema is the progressive destruction of elastic tissue, which causes loss of lung recoil and progressive hyper-expansion.

Almost two million Americans and at least three times that many individuals worldwide suffer from emphysema (see American Thoracic Society, Am. J. Resp. Crit. Care Med. 152:s77-s121, 1995). The average patient with emphysema reaches a critical level of compromise by about the age of 60 and, at that point, often begins to experience symptoms such as shortness of breath. In addition, functional capacity becomes reduced, quality of life is compromised, and the frequency of hospitalization is increased. Despite aggressive public health initiatives, cigarette smoking remains common, and emphysema will likely remain a major public health problem well into the new millennium.

Even though emphysema is a distinct condition, the therapies that have been developed to treat it are patterned after those used to treat asthma and chronic bronchitis. The treatments can be grouped into five categories: (1) inhaled and oral medications that help open narrowed or constricted airways by promoting airway muscle relaxation; (2) inhaled and oral medications that reduce airway inflammation and secretions; (3) oxygen therapy, which is designed to delay or prevent the development of pulmonary hypertension and cor pulmonale (right ventricular failure) in patients with chronic hypoxemia; (4) exercise programs that improve cardiovascular function, functional capacity, and quality of life; and (5) smoking cessation programs to delay the loss of lung function by preventing progression of smoking-related damage (Camilli et al., Am. Rev. Resp. Dis. 135:794-799, 1987). Although each of these approaches has been shown to have beneficial effects in this patient population, only oxygen therapy and smoking cessation significantly alter the natural history of this disease (Nocturnal Oxygen Therapy Trial Group, Ann. Intern. Med. 93:391, 1980).

Surgical therapy has recently been introduced as an adjunct to the medical treatments described above, and the results have been impressive. The surgical approach, known as lung volume reduction surgery (LVRS), improves lung function, exercise capacity, breathing symptoms, and quality of life in the majority of emphysema patients who meet designated selection criteria (Cooper et al., J. Thorac. Cardiovasc. Surg. 109:106-116, 1995). In LVRS, damaged, hyper-inflated lung is removed, and this is believed to provide a better fit between the over-expanded lung and the more normal sized chest wall. The fraction of the lung that remains within the chest cavity can better expand, and this increases the proportion of lung that can effectively contribute to ventilation (Fessler et al., Am. J. Resp. Crit. Care Med. 157:715-722, 1998). Recoil pressures increase, and expiratory flows improve. To date, LVRS is the only treatment that directly addresses lung hyper-expansion, which is the primary physiological abnormality of emphysema. Unfortunately, the benefits of LVRS may tend to decline over time (see Gelb et al., Am. J. Resp. Crit. Care Med. 163:1562-1566, 2001).

### SUMMARY

We have discovered that lung volume reduction, a procedure that reduces lung size by removing damaged (e.g., over-expanded) regions of the lung, can be accomplished by procedures carried out through the patient's trachea (e.g., by inserting devices and substances through a bronchoscope), rather than by procedures that disrupt the integrity of the chest wall (Ingenito et al., Am. J. Resp. Crit. Care Med. 164:295-301, 2001; Ingenito et al., Am. J. Resp. Crit. Care Med. 161:A750, 2000; Ingenito et al., Am. J. Resp. Crit. Care Med. 163:A957, 2001). We have also discovered that the methods for lung volume reduction (particularly non-surgical LVR) can be improved by damaging the epithelial cells that line the inner surface of the lung. The term “damaging” encompasses any activity that renders the population of epithelial cells less than fully or normally functional. For example, “damaging” can be achieved by disrupting, destroying, removing or ablating cells within this population (mechanically or non-mechanically (e.g., by inducing cell death)) or by otherwise rendering the cells within the epithelium less than fully functional. Preferably, the epithelial cells are selectively damaged (i.e., affected to an extent greater than, and preferably much greater than, non-epithelial cells are affected). While the methods of the present invention are not limited to those in which any particular cellular event occurs (or fails to occur), we believe the compositions and methods of the invention are most useful or successful when they inhibit one or more of the functions normally carried out by the lung epithelium. For example, the compositions and methods described herein may inhibit the ability of epithelial cells to regulate fluid passage between blood vessels and the alveolar compartment; to produce surfactant, which is critical for maintaining alveolar patency; or to serve as a barrier between the alveolar compartment and the underlying lung interstitium. While such functions help maintain homeostasis within the normal lung, we have discovered that they can hinder effective lung volume reduction (e.g., BLVR), where one aims to achieve or control scar formation. Scarring is facilitated by interstitial fibroblasts that reside beneath the epithelial surface and produce collagen. Our studies have shown that eliminating the epithelial barrier in a targeted area of the lung, in whole or in part, improves the efficacy of LVR (e.g., BLVR).

Accordingly, the present invention features methods for damaging epithelial cells within tissues, such as the lung. In some embodiments, the epithelial cells may impede a process mediated by non-epithelial cells (e.g., in the lung, epithelial cells may impede scarring, which is mediated, at least in part, by fibroblasts and which is desirable in some cases (e.g., in lung volume reduction)). Thus, the methods of the invention, or the use of the compositions described herein, can be used in any circumstance where one wishes to promote scarring or adhesion between two tissues (whether in the context of volume reduction in the lung, or to promote adhesion between damaged (e.g., traumatized) tissue in the lung or elsewhere). Epithelial cells can be damaged by administration of an enzyme, but this is far from the only means by which they can be damaged; the methods of the invention can be practiced by administering other types of agents or by applying a force that damages epithelial cells. For example, in addition to, or instead of, administering an enzyme, one could administer a pro-apoptotic agent, a photo-sensitizing agent, or some form of energy. For example, one could apply mechanical energy through small cytologic brushes; thermal energy (in the form of heat or cold); or ultrasonic energy. These methods are described further below. As noted above, regardless of the way in which the damage is caused, it can be selective (i.e., it can damage one cell type (e.g., epithelial cells) more than another cell type (e.g., a fibroblast or other non-epithelial cell); it can damage some, but not all, of the targeted cells (and, possibly, some non-targeted cells); or it can damage essentially all of the targeted cells to a limited extent), and it can be characterized in several ways (e.g., as selective ablation or controlled shedding).

As the methods for damaging the epithelial cell lining can be carried out as part of a lung volume reduction procedure, the invention also features methods of reducing lung volume by administering, to a patient (which includes but is not limited to human patients; domesticated animals may also be treated), an agent that damages epithelial cells, and compositions (e.g., physiologically acceptable compositions comprising one or more such agents) are also within the scope of the present invention. No special meaning is attached to the term “agent.” Unless otherwise noted, it is interchangeable with other terms such as “substance” or “compound,” and it can be biologically active (such as an enzyme) or inactive (such as a compound that is inert until activated by subsequent application of, for example, heat, cold, or some form of light; the substance can also be a prodrug). More specifically, the substance can be an enzyme (e.g., a protease such as a serine protease such as trypsin, chymotrypsin, elastase, or a matrix metalloproteinase; mixtures of enzymes can also be used). Thus, in one embodiment, the invention features a method of reducing lung volume by administering, through the patient's trachea, a composition comprising an enzyme. This step can be followed (immediately or after one or more intervening steps which may serve to contain or limit the enzyme's activity) by a procedure that induces collapse of a region of the lung in which epithelial cells have been damaged (exemplary intermediate steps are described below). For example, one can induce collapse by administering a material that increases the surface tension of fluids lining the alveoli (i.e., a material that acts as an anti-surfactant). This material can be introduced through a bronchoscope (preferably, through a catheter or similar device lying within the bronchoscope), and it can include fibrinogen, fibrin (e.g., a fibrin I monomer, a fibrin II monomer, a des BB fibrin monomer, or any mixture or combination thereof), or biologically active mutants (e.g., fragments) thereof. In the event fibrinogen is selected as the anti-surfactant, one can promote adhesion between collapsed areas of the lung by exposing the fibrinogen to a fibrinogen activator, such as thrombin (or a biologically active variant thereof), which cleaves fibrinogen and polymerizes the resulting fibrin. Other substances, including thrombin receptor agonists and batroxobin, can also be used to activate fibrinogen. If fibrin is selected as the anti-surfactant, no additional substance need be administered; fibrin can polymerize spontaneously, thereby adhering one portion of the collapsed tissue to another.

When the tissue in question is lung tissue, tissue collapse can also be induced by impeding airflow into and out of the region of the lung that is targeted for collapse. This can be achieved by inserting a balloon catheter through, for example, a bronchoscope and inflating the balloon so that it occludes the bronchus or bronchiole into which the balloon portion of the catheter has been placed. Devices other than a balloon catheter may also be used so long as they can be maneuvered into the desired location within the respiratory tract and they can create a barrier that impedes airflow to alveoli (or any portion of the lung distal to the occlusion). The barrier can be temporary (i.e., sustained only as long as is necessary for distal lung tissue to collapse) or more permanent (e.g., a plug of degradable or non-degradable material).

Any of the compositions administered to the patient (e.g., an enzyme-containing solution or an anti-surfactant) can also contain one or more antibiotics to help prevent infection. Alternatively, or in addition, antibiotics can be administered via other routes (e.g., they may be administered orally or intramuscularly). Any of the compositions administered to the patient can also be included in a kit. For example, the invention features kits that include an enzyme-containing preparation (e.g., a physiologically acceptable solution that contains one or more serine proteases) and/or a preparation to inhibit the activity of the protease (e.g., a physiologically acceptable solution that contains serum or a neutralizing antibody) and/or a preparation to induce lung collapse (e.g., a physiologically acceptable solution that contains an anti-surfactant) and/or an antibiotic. These preparations can be formulated in accordance with the information provided further below and with knowledge generally available to those who routinely develop such preparations. The preparations can be sterile or contained within vials or ampules (or the like; in solution or in a lyophilized form) that can be sterilized, and the preparations can be packaged with directions for their preparation (if required) and use. A kit containing the preparations just described would be useful when one wishes to use enzymes to damage epithelial cells within the lung prior to a lung volume reduction procedure. The enzyme and/or the preparation that inhibits the enzyme's activity can also be packaged with other agents. For example, they can be packaged with nucleic acids (those that encode polypeptides, antisense oligonucleotides, or an siRNA) that can be used to transfect mesenchymal or other cell types remaining within the lung after the epithelial cells have been damaged, or with other therapeutic agents (e.g., polypeptides or small molecules). The invention also features kits that would be used when one wishes to condition the lung in other ways. For example, where one wishes to use a photodynamic therapy, the kit can contain liposomes and a photodynamic agent such as photofrin (liposome-encapsulated photodynamic agents per se are also within the scope of the invention); where one wishes to use a mechanical device, the kit may contain a cytology brush configured to extend to and remove epithelial cells from a targeted region of the respiratory tract (the brush per se is also within the scope of the invention); where one wishes to use ultrasonic energy, the kit may contain a perfluorocarbon; and where one wishes to use electric energy, the kit may contain an electrolyte solution to improve energy conduction and a rinsing agent to dilute the electrolyte solution after use. Any of these kits can contain devices used in non-surgical lung volume reduction. For example, they can also contain a catheter (e.g., a single- or multi-lumen (e.g., dual-lumen) catheter that, optionally, includes a balloon or other device suitable for inhibiting airflow within the respiratory tract), tubing or other conduits for removing material (e.g., solutions, including those that carry debrided epithelial cells) from the lung, and/or a bronchoscope.

As with the enzyme-containing kits, those designed to condition the epithelium in other ways can include agents useful in procedures other than lung volume reduction. For example, they can contain nucleic acids (those that encode polypeptides, antisense oligonucleotides, or an siRNA) that can be used to transfect mesenchymal or other cell types remaining within the lung after the epithelial cells have been damaged, or other therapeutic agents (e.g., polypeptides or small molecules).

The methods in which epithelial cells are damaged can also be carried out as part of other therapeutic regimes. They can be carried out, for example, when one wishes to deliver a therapeutic agent (e.g., a nucleic acid molecule, a protein, or a chemical compound (e.g. a small molecule)) to cells that lie beneath (or are otherwise obscured by) epithelial cells. Accordingly, the invention features methods of delivering a therapeutic agent to a cell within a patient, wherein the cell is a non-epithelial cell that lies beneath an epithelial cell layer, or is otherwise obscured by an epithelial cell. The methods can be carried out by, first, damaging the epithelial cells by any of the methods, mechanical or non-mechanical, described herein and, second, administering a therapeutic agent to the region where the epithelial cells were damaged. The damage can include destroying epithelial cells and the destruction is preferably selective (i.e., the epithelial cells are affected to an extent greater than, and preferably much greater than, non-epithelial cells are affected). The step in which a therapeutic agent is administered can be carried out by any method known in the art. When epithelial cells are damaged in preparation for delivering a therapeutic agent (including an agent that induces lung collapse as part of a lung volume reduction procedure), the extent of the damage to the epithelial cells can vary. It is not necessary to destroy all epithelial cells. The method will be considered a success so long as the outcome is better than the outcome reasonably expected without any epithelial cell ablation or damage.

More specifically, the invention includes methods for performing non-surgical lung volume reduction in a patient by (a) administering, through the patient's trachea, a composition comprising an enzyme (e.g., a protease, such as a serine protease (e.g., trypsin, chymotrypsin, elastase, or an MMP); and (b) collapsing a region of the lung, at least a portion of which was contacted by the composition administered in step (a). The patient can have COPD (e.g., emphysema) or their lung can be damaged by a traumatic event. The tissue in the targeted area can also include an abscess or fistula. One can similarly treat other tissues (i.e., non-lung tissues) by exposing those tissues to an enzyme-containing composition (or other composition described herein). These tissues may be those that are obscured from a therapeutic agent by epithelial cells or that will contact an implantable device. Where the lung is targeted, one can collapse a region of the lung by administering, to the targeted region, a substance that increases the surface tension of fluids lining the alveoli in the targeted region, the surface tension being increased to the point where the region of the lung collapses. The concentration of the active agents in the compositions of the invention are described further below, but we note here that the concentrations will be sufficient to damage the epithelial cell lining of the lung or the epithelium lining or otherwise covering another tissue. The compositions described herein can be used not only for lung volume reduction and other tissue treatments, but also for use as medicaments, or for use in the preparation of medicaments, for treating patients who have a disease or condition that would benefit from selective epithelial damage and subsequent fibrosis or scar formation (e.g., a disease or condition in which the target cells would otherwise be obscured by the epithelial lining of a tissue or one that can be treated with an implanted device (e.g., a valve, pump, or prosthetic device)).

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are schematic representations of a mechanical method for damaging epithelial cells, which may be done to condition a region of the lung prior to BVR (bronchoscopic volume reduction) or prior to administering a therapeutic agent to cells beneath the epithelial layer. FIG. 1A illustrates insertion of a device in which an elongated flexible member (e.g., a wire or cable) is attached to a brush that is guided through a bronchoscope into a region of a patient's respiratory tract that is targeted for reduction. The brush shown here has unidirectional bristles to facilitate removing epithelial cells. FIG. 1B illustrates the juxtaposition between the brush and the epithelial cells in more detail before (left-hand panel) and after (right-hand panel) the cells are treated. Fibroblasts lie beneath an epithelial cell layer that is contacted by the brush. As the brush is withdrawn (and it may be inserted and withdrawn over a region several times (i.e., the procedure may involve a scrubbing-type action)) the bristles damage and/or remove the epithelial cells. As a result, epithelial cells are dislodged and may become trapped in the bristles of the brush. The epithelial cell layer is then wholly or partially denuded.

FIGS. 2A and 2B are schematic representations of a method for damaging epithelial cells within the lung using ultrasonic energy. FIG. 2A illustrates insertion of a balloon-tip dual lumen catheter through a bronchoscope to a region of the patient's lung that is targeted for reduction. When the balloon is inflated, it isolates the target region. The catheter and the target region of the lung contain a medium such as a perfluorocarbon (PFC) medium. FIG. 2B illustrates the application of ultrasonic energy in more detail. An ultrasonic generator is attached to the proximal end of the PFC-filled cathether, and ultrasound energy is transmitted to the epithelial cell layer (left-hand panel). Following application of the ultrasonic energy (right-hand panel), the epithelial cell layer is denuded. Detached cells and the PFC medium can be removed by suction (e.g., a suction tube can be inserted through the second of the two lumens in the dual lumen catheter). This method, or any of the others for damaging epithelial cells, may be done to condition a region of the lung prior to lung volume reduction or prior to administering a therapeutic agent to cells beneath the epithelial layer.

FIGS. 3A and 3B are schematic representations of a method for damaging epithelial cells within the lung using thermal energy. FIG. 3A illustrates insertion of an insulated cryocatheter, through which one can administer cold nitrogen gas to a region of the patient's lung that is targeted for reduction. When the balloon is inflated, it isolates the target region. Suction may be applied for a time sufficient to degas the region (e.g., 3-4 minutes) before the N₂ is applied, and the process may be repeated (i.e., the tissue may be thawed or allowed to thaw before N₂ is again applied). FIG. 3B illustrates the application of cold gas in more detail (left-hand panel). Epithelial cells detach following the freeze-thaw process (right-hand panel).

FIGS. 4A and 4B are schematic representations of a method for damaging epithelial cells within the lung using electrical energy. FIG. 4A illustrates an expansion-tipped unipolar electrode catheter positioned within a selected (or target) region of the lung. A solution containing electrolytes (an “electrolyte rinse solution”) can be placed in the targeted region of the lung to improve energy conduction distal to the electrode. The structure of the catheter is shown in more detail in FIG. 4B. A wire is contained within the flexible shaft of the catheter and an electrode resides at or near the tip. The arrows within the airways represent energy transmitted from a power source and through the rinse solution (left-hand panel). The epithelial cell layer is damaged when electrical energy is applied; some of the epithelial cells that are dislodged are shown within the airway (right-hand panel). These cells can be removed by removing the rinse solution (e.g., with a suction device inserted through the bronchoscope or a lumen of the catheter).

FIGS. 5A and 5B are schematic representations of a method for damaging epithelial cells within the lung using a photodynamic therapy (PDT). FIG. 5A illustrates a balloon-tipped dual lumen catheter positioned within a targeted region of the lung. A PDT-compatible solution, such as one containing liposomes and photofrin, is contained within the target region. To activate the photofrin and damage epithelial cells, a light-emitting fiber is extended through a lumen of the catheter (FIG. 5B, left-hand panel). The epithelial cells that slough away from the layer of epithelial cells can be removed by removing the photofrin solution (e.g., with a suction device inserted through the bronchoscope or a lumen of the catheter). (Not shown here but also within the scope of the present invention is pretreatment using systemic application of photofrin rather than the liposomal photofrin solution.)

FIGS. 6A and 6B are schematic representations of a method for damaging epithelial cells within the lung using an enzyme-containing solution. FIG. 6A illustrates a single lumen catheter (although a multi-lumen catheter can also be used), inserted through an instrumentation (or “working”) channel of a bronchoscope and into the target region of the lung. A balloon inflated at the distal tip of the catheter seals the target region. The enzyme-containing solution is applied first and a solution containing a substance that inhibits the activity of the enzyme may be applied subsequently (FIG. 6B, left-hand panel). Epithelial cells that are sloughed off may be removed by lavage after either the enzyme-containing solution or the neutralizing solution is applied (FIG. 6B, right-hand panel).

FIG. 7 is a line graph showing the relationship between lung volume (liters) and Ptp (cm H₂O) in untreated animals (solid line; baseline) and those treated with papain to model emphysema (dotted line; emphysema). There is a significant increase in lung volume (measured by plethysmography) in the papain-treated animals, which demonstrates hyperinflation as a result of tissue damage.

FIG. 8 is a bar graph showing lung volume (liters; VC=vital capacity; RV=residual volume) in untreated animals (Baseline), papain-treated animals (Emphysema), and following treatment by enzyme pre-conditioning and BVR (Post BVR). These data demonstrate hyperinflation as a result of tissue damage, and a return to normal volumes after BVR.

FIG. 9 is a pair of images of the chest cavity before treatment with papain (left-hand image) and after papain treatment (right-hand side). A 5 cm bullous lesion is apparent after papain treatment.

FIG. 10 is a series of drawings showing the effect of enzyme pre-conditioning on epithelial cells within the lung. The top panel illustrates the epithelial surface in cross-section in an untreated animal. Fibroblasts lie beneath the epithelial cell layer. The middle panel illustrates a disruption in the epithelial cell layer, resulting from exposure to an enzyme. Subsequently (e.g., after application of a hydrogel), mesenchymal cells can migrate into the airway lumen and promote scar formation. As shown in the bottom panel, chemotaxis of fibroblasts and subsequent collagen deposition leads to scarring of the target region, which secures the area of collapse.

FIG. 11 is a bar graph illustrating lung resistance (cm H₂O/L/sec) before and after induction of emphysema by papain treatment. Compared to baseline (grey shading), post-papain-treated animals demonstrated an increase in total lung resistance of 40±9%, and an increase in airway resistance of 75±16%.

FIG. 12 is a bar graph illustrating lung volumes (in liters) in healthy animals (black bar; baseline) in animals treated with papain (grey bar; emphysema), and after enzyme pre-conditioning and BVR (white bar; Post BVR). Total lung capacity (TLC), the total volume within the lung, increased 10±3%, the residual volume (RV), the trapped gas within the lung, decreased 66±21%, and vital capacity (VC), the functional volume within the lung increased 11±4%.

FIG. 13 is a Campbell diagram of baseline physiology, after induction of emphysema by papain treatment, and after enzyme pre-conditioning/BVR (see the legend; volume (liters) vs. Ppl (cm H₂O)). The diagram demonstrates the inter-relationship between chest wall and lung mechanics that ultimately determine the static properties of the respiratory system. Papain-induced emphysema had no significant impact on either active (CWa) or passive (CWp) chest wall mechanics, but caused a significant increase in both total lung capacity (TLC) and RV.

FIGS. 14A and 14B are images of the respiratory system at various times. FIG. 14A shows a CT scan of an animal with heterogeneous emphysema, with a bullous lesion developed in response to papain instilled bronchoscopically (left-hand panel). The bullae in the right upper dorsal lobe measured 5×3×7 cm before treatment. After enzyme pre-conditioning and BVR (right-hand panel), the lesion was reduced in size to 3×2×2 cm. FIG. 14B shows a CT scan of an animal with heterogeneous emphysema, with a 5 cm bullae (upper panel) that was completely closed three months after the BVR procedure was performed (lower panel). In addition, sites of diffuse emphysema treated with BVR are also visible.

FIG. 15 is a Table summarizing the physiological parameters measured in post-BVR studies performed at 1 and 3 months (see the Examples).

### DETAILED DESCRIPTION

The present invention features methods that can be used to damage (e.g., to selectively ablate) epithelial cells (e.g., those in an epithelial cell layer) in an organ, such as the lung. The damage can be done in the context of another procedure. For example, it can be done in preparation for reducing the volume of inherently collapsible tissue; in preparation for treatment of cells that would otherwise be obscured by the epithelial lining of a tissue; or in preparation for processes where one epithelial cell-bearing tissue is fused to another or to an implanted device (e.g., a valve, pump, or prosthetic device).

When carried out in the context of lung volume reduction (e.g., non-surgical LVR), the methods for effecting epithelial damage can be used to treat patients who have certain diseases of the lung, such as emphysema (a chronic obstructive pulmonary disease (COPD)). While it may seem counterintuitive that respiratory function would be improved by removing part of the lung, excising over-distended tissue (as seen in patients with heterogeneous emphysema) allows adjacent regions of the lung that are more normal to expand. In turn, this expansion allows for improved recoil and gas exchange. Even patients with homogeneous emphysema benefit from LVR because resection of abnormal lung results in overall reduction in lung volumes, an increase in elastic recoil pressures, and a shift in the static compliance curve towards normal (Hoppin, Am. J. Resp. Crit. Care Med. 155:520-525, 1997).

BLVR is performed by, for example, collapsing a selected region of the lung and adhering one portion of the collapsed region to another by promoting fibrosis or scarring in or around the adherent tissue. It is advantageous to prepare (or “condition”) one or more of the affected regions of the lung or a portion thereof. The conditioning, which promotes fibrosis and can lead to stronger or longer-lasting adhesion between collapsed portions of the tissue, can be carried out in a number of ways. Various methods for conditioning tissue, any of which can be carried out prior to a lung volume reduction (e.g., BLVR) or another of the therapeutic procedures described herein, are described below. Moreover, these methods may be combined. For example, one could use an enzyme and ultrasonic energy to remove epithelial cells from the respiratory tract.

### Methods that Employ an Enzyme

One can use a preparation (e.g., a physiologically acceptable solution, suspension, or mixture; exemplary formulations are described further below) that contains one or more enzymes to selectively damage epithelial cells (e.g., epithelial cells lining the respiratory tract). Preparations that contain trypsin but lack divalent cations are used in conventional cell culture practice to displace cells, including epithelial cells and fibroblasts, from tissue culture plastic. Such preparations have also been used in situ to prepare primary epithelial cell cultures; they are known as an effective means for removing the epithelial cell layer without causing marked damage to the tissue as a whole.

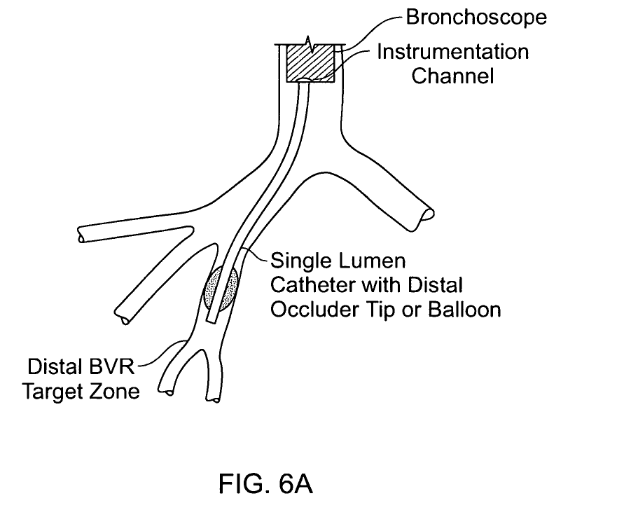

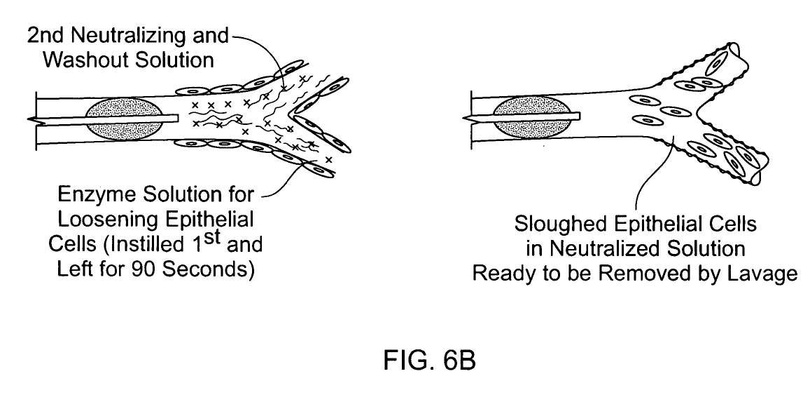

The studies described below demonstrate that these preparations are among those effective in selectively ablating epithelial cells (the studies are performed in a large animal model of emphysema). Accordingly, the invention features methods in which proteases are used to disrupt epithelial cell attachment to the underlying sub-epithelial interstitium and basement membrane (FIGS. 6A and 6B), followed further by a therapeutic process (e.g., lung volume reduction (e.g., BLVR) or administration of a therapeutic agent to a cell that was previously at least partially obscured by an epithelial cell). The invention also features physiologically acceptable compositions that include one or more agents (e.g., proteases; see below)) that disrupt the attachment between epithelial cells and surrounding or underlying cell types (e.g., subepithelial interstitium and/or basement membranes) for use as medicaments or for use in the preparation of medicaments for treating patients who have COPD (e.g., emphysema) or another disease or condition that would benefit from selective epithelial damage and subsequent fibrosis or scar formation (e.g., a disease or condition in which the target cells would otherwise be obscured by the epithelial lining of a tissue or one that can be treated with an implanted device (e.g., a valve, pump, or prosthetic device)).

A variety of different proteases, including serine proteases, can be used. Serine proteases are a superfamily of enzymes that catalyze the hydrolysis of covalent peptidic bonds. In the case of serine proteases, the mechanism is based on nucleophilic attack of the targeted peptidic bond by a serine. Cysteine, threonine or water molecules associated with aspartate or metals may also play this role. In many cases, the nucleophilic property of the group is improved by the presence of a histidine, held in a “proton acceptor state” by an aspartate. Aligned side chains of serine, histidine and aspartate build the catalytic triad common to most serine proteases.

There are approximately 700 serine proteases, grouped into 30 families, and further grouped into 5 clans. Representative members of these families, any of which can be used in the methods described herein (and any of which can be used for the manufacture of a medicament for use in treating a patient who has COPD (e.g., emphysema) or another condition which would benefit from controlled epithelial cell damage), include trypsin, chymotrypsin, alpha-lytic endopeptidase, alpha-lytic endopeptidase, glutamyl endopeptidase (V8), protease Do (htrA) (Escherichia), togavirin, lysyl endopeptidase, IgA-specific serine endopeptidase, flavivirin, hepatitis C virus NS3 endopeptidase, tobacco etch virus 35 Kd endopeptidase, cattle diarrhea virus p80 endopeptidase, equine arteritis virus putative endopeptidase, apple stem grooving virus serine endopeptidase, subtilases, subtilisin, kexin, tripeptidyl-peptidase II, prolyl oligopeptidase, prolyl oligopeptidase, dipeptidyl-peptidase IV, acylaminoacyl-peptidase, carboxypeptidase C, lactococcus X-Pro dipeptidyl-peptidase, lysosomal Pro-X carboxypeptidase, D-Ala-D-Ala peptidase family 1, D-Ala-D-Ala peptidase family 2, D-Ala-D-Ala peptidase family 3, ClpP endopeptidase, endopeptidase La (Lon), LexA repressor, bacterial leader peptidase I, eukaryote signal peptidase, omptin, coccidiodes endopeptidase, and assemblin (Herpesviruses protease). The invention also features physiologically acceptable compositions that include one or more of these enzymes for use as medicaments or for use in the preparation of medicaments for treating patients who have a disease or condition that would benefit from selective epithelial damage and subsequent fibrosis or scar formation (e.g., a disease or condition in which the target cells would otherwise be obscured by the epithelial lining of a tissue or one that can be treated with an implanted device (e.g., a valve, pump, or prosthetic device)).

Enzymatic preparations are described further below. We note here, however, that the concentration of the enzyme(s) within the preparation can be readily determined by one of ordinary skill in the art and will be such that the epithelial cell lining will be damaged (e.g., by loss of epithelial cells) but the cells (e.g. mesenchymal cells) under that lining will be substantially unaffected (in the lung, the underlying cells will not be so affected that they cannot mediate fibrosis). This can be determined by, for example, histological analysis or by assessing outcome (e.g., if there is no indication of fibrosis, the enzyme treatment may have destroyed the underlying fibroblasts, indicating that the concentration of the enzyme or the length of the treatment is excessive). Such determinations can be made in large animal models before human clinical trials.

When trypsin is included in the preparation, it can be present as 0.1-10.0% (w/v) of the solution (e.g., 0.1-9.0%, 0.1-8.0%, 0.1-7.0%, 0.1-6.0%, 0.1-5.0%, 0. 1-4.0%, 0.1-3.0%, 0.1-2.0%, 0.1-1.0%, 0.2-0.8%, 0.2-0.5%, or about 0.1%, 0.2%, 0.5%, 0. 8% or 1.0%, or about 5.0-10.0%, 6.0-10.0%, 7.0-10.0%, 8.0-10.0%, or 9.0-10.0%).

When collagenase (e.g., Type I collagenase) is included in the preparation (e.g., as for any of the other compositions described herein, a physiologically acceptable composition useful for treating a patient who has COPD (e.g., emphysema) or in the manufacture of a medicament for use in treating such a patient), it can be present in the same percentage ranges given above for trypsin. Alternatively, one can include 50-100 U/ml of collagenase (e.g., 50-90, 50-80, 50-70, 50-60, 60-90, 70-90, 80-90, or 90-100 U/ml). When disspase is included in the preparation, it can be present in the same percentage ranges given above for trypsin. Alternatively, one can include 0.6-2.4 U/ml of disspase (e.g., 0.6-2.0, 0.6-1.8, 0.6-1.6, 0.6-1.4, 0.6-1.2, 0.6-1.0, 0.6-0.8, 0.8-1.0, 0.8-1.2, 1.0-2.0, 1.2-1.8, or 1.4-1.6 U/ml). When elastase is included in the preparation, it can be present in the same percentage ranges given above for trypsin. Alternatively, one can include 0.1-1.0 mg/ml elastase (e.g., 0.1-0.9, 0.2-0.8, 0.3-0.7, 0.4-0.6, about 0.5, 0.1-0.2, 0.1-0.3, 0.1-0.4, 0.1-0.5, 0.5-1.0 or 0.5-0.8 mg/ml). When chymotrypsin is included in the preparation, it can be present in the same percentage ranges given above for trypsin. Alternatively, one can include 0.1-1.0 mg/ml elastase (e.g., 0.1-0.9, 0.2-0.8, 0.3-0.7, 0.4-0.6, about 0.5, 0.1-0.2, 0.1-0.3, 0.1-0.4, 0.1-0.5, 0.5-1.0 or 0.5-0.8 mg/ml chymotrypsin).

The enzyme-containing preparation can be removed from the area if desired by, for example, suction or with an absorbent material. In the event the preparation is administered to a region within the lung, it can be applied through a catheter inserted through the working channel of a bronchoscope, and removed by subsequently inserting a suction tube through the catheter. To contain the solution (and this is true of any of the solutions described herein) within a particular region of the lung, one can use a balloon-tipped catheter; when the balloon is inflated, it occludes the passageway to the distal portions of the lung.

The enzyme-containing preparation can also be affected by applying a neutralizing solution that inhibits the activity of the enzyme used (inhibition need not be complete in order for the neutralizing solution to be effective). The neutralizing solution can include a protein (e.g., an antibody) that specifically binds the enzyme and thereby inhibits its functional activity or it can include a nonspecific agent, such as serum and/or aprotinin.

Any of the enzyme-containing compositions described here can be formulated as physiologically acceptable compositions that can be used to treat, or used in the preparation of a medicament to treat, patients who have COPD (e.g., emphysema) or another disease or condition that would benefit from selective epithelial damage and subsequent fibrosis or scar formation (e.g., a disease or condition in which the target cells would otherwise be obscured by the epithelial lining of a tissue or one that can be treated with an implanted device (e.g., a valve, pump, or prosthetic device).

### Methods that Employ Mechanical Force

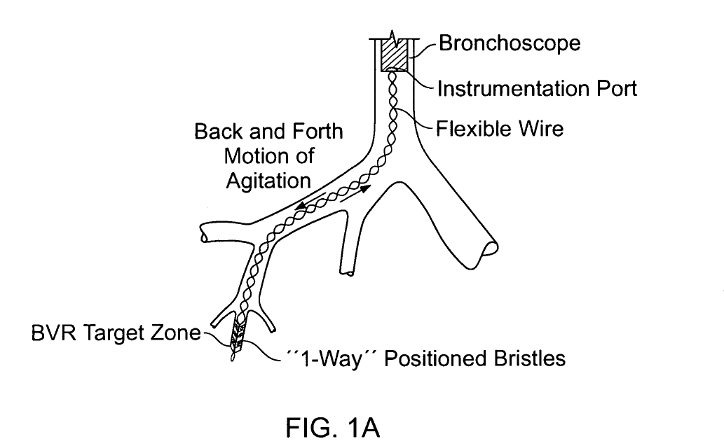

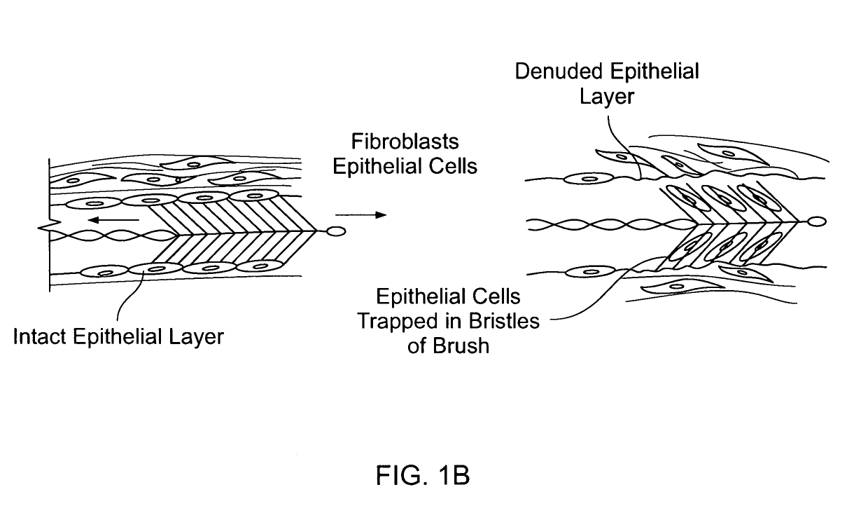

In addition to, or as an alternative to, the chemical (e.g., enzymatic) treatments described herein, tissue (e.g., lung tissue) can be exposed to a mechanical force that damages the epithelium. For example, one can simply brush or otherwise abrade the selected region with, for example, a cytology brush specifically designed for the organ in question. For example, the brush can include short bristles that are capable of de-epithelializing a particular region of the airway in preparation for non-surgical (e.g., bronchoscopic) volume reduction therapy (FIG. 1). This embodiment can include the use of a small (1.5-2.0 mm) brush that can be passed into multiple small airways of the projected target region and gently rubbed to remove the selected cells (brushes having an outer diameter of 2-5 mm can be obtained from Bard Endoscopy and U.S. Endoscopy; other commercial suppliers and other brushes are readily available).

If desired, the epithelial cells that have been removed (i.e., ablated) from the target region can be washed away by administering a physiologically compatible solution (e.g., saline or a buffered solution such as phosphate-buffered saline). The “rinsing agent” can be applied through a catheter or tube inserted through a working channel of the bronchoscope and removed by applying suction to the same or a different device inserted into the target region (more generally, and regardless of the manner in which epithelial cells are ablated, those cells can be removed from the target region before a therapeutic procedure is carried out or a therapeutic agent is administered). An anti-surfactant (e.g. fibrin or fibrinogen, or a detergent), suction, or a mechanical blockade of the airway can then be applied to induce regional collapse (the collapsed region containing at least some portions in which the epithelial lining was damaged). As following other methods of inducing epithelial damage and regional collapse, a reagent such as a fibrin-based hydrogel can be applied to promote scar formation and improve the strength or duration of the collapse.

### Methods That Employ Ultrasonic Energy

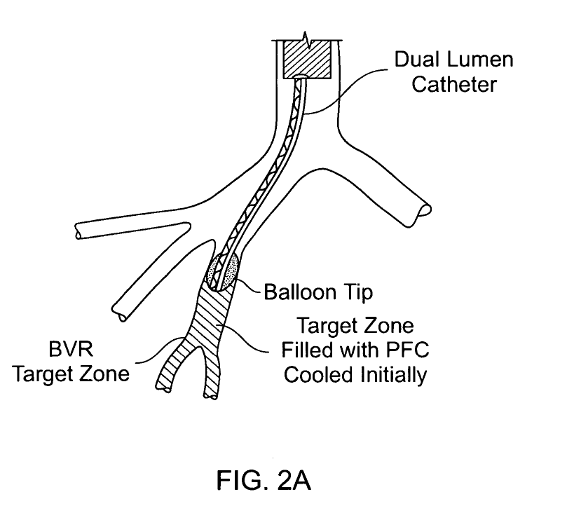

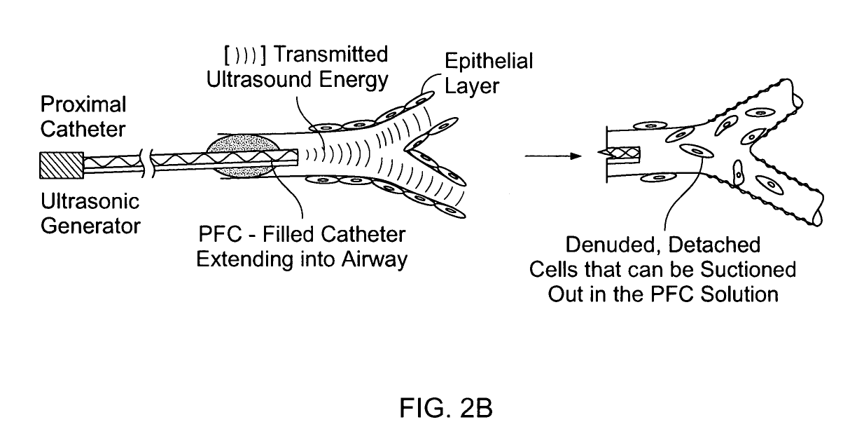

In addition to, or as an alternative to, enzymatic treatment, tissue (e.g., lung tissue) can be exposed to ultrasonic energy that damages the epithelium. Sonication is a biophysical technique that is frequently used in cell and molecular biology to disrupt cell membranes (see, e.g., Hunter and Hanrath, Thorax 47:565, 1992). In the context of the present invention, focused ultrasonic energy is applied selectively to the epithelial surface to damage (e.g., remove cells from) the epithelial layer. The specific target organ or a region thereof (e.g., all or part of an over-inflated region of the lung) can be filled with (or can include) a liquid carrier reagent that is excited with an ultrasonic probe (the ultrasonic source being at a proximal location). The carrier reagent can be a high-density perfluorocarbon, which facilitates oxygen and carbon dioxide transport and readily transmits ultrasonic energy (FIGS. 2A and 2B). The carrier reagent, and any epithelial cells contained within it, can be removed (by, for example, suction). If desired, the affected region can also be rinsed with a physiologically compatible solution (e.g., saline or a buffered solution such as phosphate-buffered saline). The “rinsing agent” can be applied through a catheter or tube inserted through a working channel of the bronchoscope and removed by applying suction to the same or a different device inserted into the target region. As following other methods of inducing epithelial damage, an anti-surfactant (e.g. fibrin), suction, or a mechanical blockade of the airway can then be applied to induce regional collapse (the collapsed region containing at least some portions in which the epithelial lining was damaged). As following other methods of inducing epithelial damage and regional collapse, a reagent such as a fibrin-based hydrogel can be applied to promote scar formation and improve the strength or duration of the collapse.

### Methods That Employ Thermal Energy

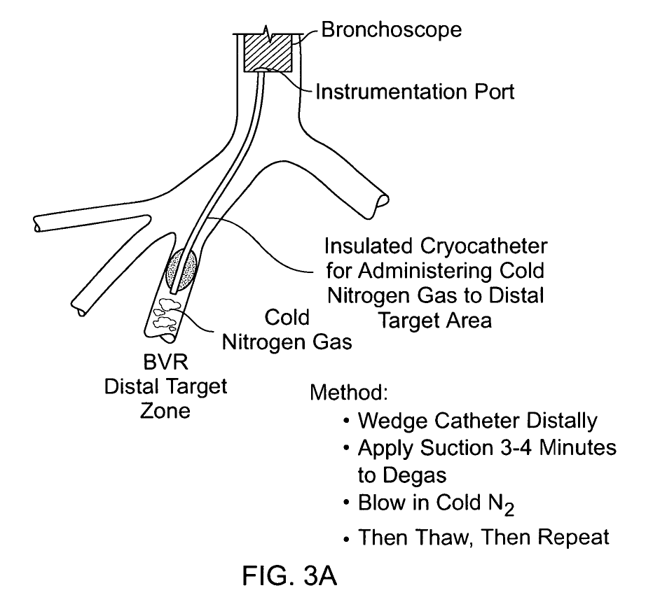

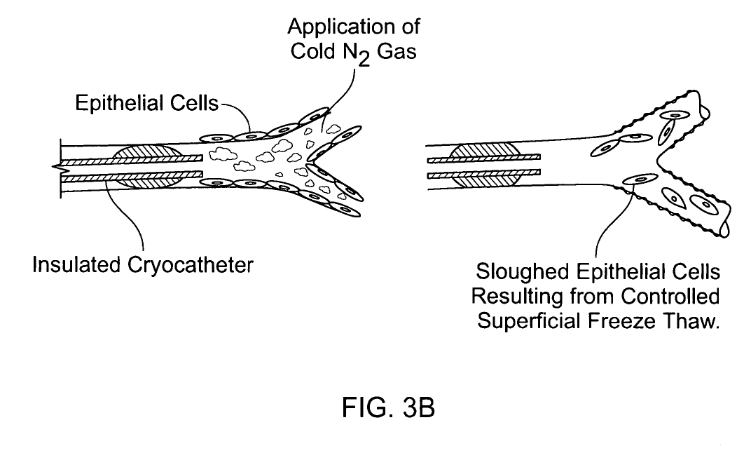

In addition to, or as an alternative to, other methods for damaging the epithelium, tissue (e.g., lung tissue) can be exposed to thermal energy (heat or cold) that damages the epithelium (see FIGS. 3A and 3B). For example, both heat, applied as laser energy, and cold applied via a cryoprobe have proven effective in “necrosing” endobronchial lesions, primarily cancers. Cryoprobes that are identical to or similar to those currently used could be applied to cause superficial damage to target regions of lung (see, e.g., Angel, Cryotherapy and electrocautery in the management of airway tumors, presented in: Multimodality management of tumors of the aerodigestive tract. Boston, Mass., November 2-3). Epithelial cells are more susceptible to damage by freeze-thaw cycles than are interstitial cells. If desired, the affected region can be rinsed with a physiologically compatible solution, as described above, to remove epithelial cells that have become dislodged, and an anti-surfactant (e.g. fibrin), suction, or a mechanical blockade of the airway can then be applied to induce regional collapse (the collapsed region containing at least some portions in which the epithelial lining was damaged). As following other methods of inducing epithelial damage and regional collapse, a reagent such as a fibrin-based hydrogel can be applied to promote scar formation and improve the strength or duration of the collapse.

### Methods That Employ Electric Energy

In addition to, or as an alternative to, other methods for damaging the epithelium, tissue (e.g., lung tissue) can be exposed to an electric current using pre-selected energy levels and waveform patterns. The energy can be delivered to a selected region of the lung in a manner that causes epithelial cells to dislodge from the underlying basement membrane. Preferably, the current is applied so that adjacent tissues are not significantly injured (see Angel, supra). To modulate (e.g., increase the effectiveness of) current delivery within target areas of lung, an electrolyte solution may be administered to those areas. This solution will wash out at least some of the naturally occurring surfactant within the lung, which contains lipids that limit energy transmission by acting as an insulator. The solution also acts as a chemical conduction system to further improve energy delivery. The solution can be administered and withdrawn (by, for example, suction) before the electrical current is applied; the residual layer serves as a sufficient conducting medium and improves energy transmission distal to the proximal current source.

The precise pattern of energy delivery may vary, depending upon whether proximal or distal de-epithelialization is desired. One of ordinary skill in the art would be able to determine the optimal pattern of energy to use to dislodge cells without causing significant injury. Programmable analog waveform generators, or computerized digital wave generators may be used to deliver any of a variety of different patterns.

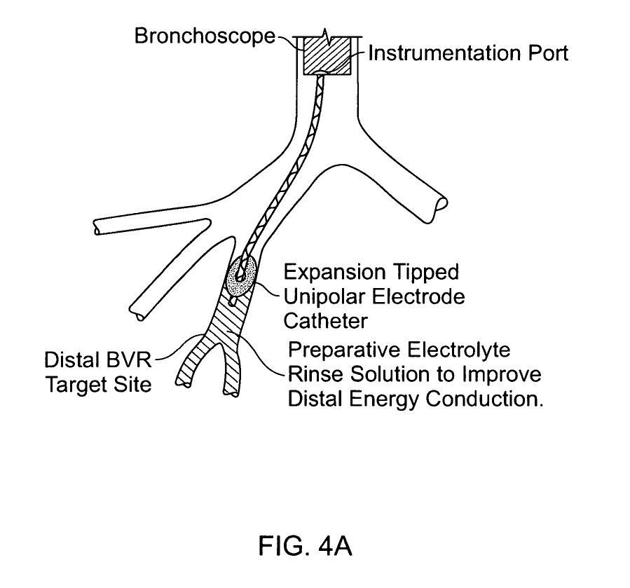

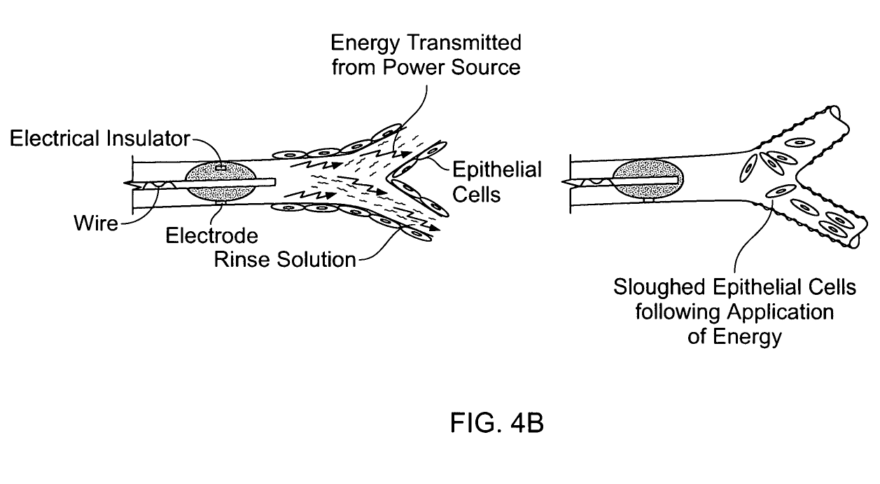

A unipolar catheter electrode may be used to transmit energy from the programmable energy source outside the patient distally into the lung. The electrode should be designed such that it is thin and flexible enough to fit through the channel of a fiber optic bronchoscope (FIGS. 4A and 4B). The purpose of the system is to transmit energy along the airway surface. Thus the conducting superficial electrode is circumferentially located, and positioned at the tip of the catheter to allow for insertion distally into the patient.

As following other methods of inducing epithelial damage, an anti-surfactant (e.g. fibrin), suction, or a mechanical blockade of the airway can be applied after the electric current to induce regional collapse (the collapsed region containing at least some portions in which the epithelial lining was damaged). As following other methods of inducing epithelial damage and regional collapse, a reagent such as a fibrin-based hydrogel can be applied to promote scar formation and improve the strength or duration of the collapse.

### Methods That Employ Photo-sensitizing Agents

In addition to, or as an alternative to, other methods for damaging the epithelium, tissue (e.g., lung tissue) photodynamic therapy (PDT) can be used to selectively ablate epithelial cells. PDT has proven clinically effective in generating targeted endobronchial tissue death (Pass, J. Natl. Cancer Inst. 85:443, 1993). This approach uses systemic therapy with a photosensitizing agent known as photophrin, a compound that is readily taken up by cells and renders them sensitive to light energy at a specific wavelength. The fluorescent properties of this intracellular dye result in tissue damage at sites wherever the monochromatic sensitizing light source is directed. As a result, site-specific endobronchial tissue injury can be generated. Accordingly, the invention features use of photodynamic or photo-sensitive agents (e.g., photophryin) for the manufacture of a medicament for use in treating a patient who has COPD (e.g., emphysema)

At present, PDT utilizes systemic photophrin exposure; site specificity is accomplished by carefully directed light application, and the present invention includes photodynamic preconditioning methods wherein the photophrin has been administered systemically. However, the invention also features methods in which a photo-sensitive agent (e.g., photofrin) is administered to the lung by way of a bronchoscope. Such localized application has advantages in that the patient is not required to remain in the dark for any period of time; with systemic administration, patients must avoid exposure to light until the photophrin is no longer present in active amounts. Localized administration (e.g. administration under bronchoscopic guidance) thus allows for greater control of photosensitivity. Optionally, the photo-sensitive agent can be mixed with or encapsulated within liposomes by methods known in the art prior to administration to a patient. The liposomal mixture may facilitate endobronchial spreading. Without limiting the invention to methods achieved by any particular cellular mechanism, the liposomal particles may be taken up by endocytosis into epithelial cells by the same pathway that is involved in surfactant recycling. Thus, the present invention also relates to photodynamic preconditioning methods wherein the photophrin has been administered selectively via liposomal delivery, and to the use of liposome-associated photodynamic or photo-sensitive agents for the manufacture of a medicament for use in treating a patient who has COPD (e.g., emphysema). As noted in connection with other epithelial cell damaging-agents described above, these compositions are also useful in treating patients (or in the preparation of a medicament for treating patients) who have suffered a traumatic injury; patients whose target cells are obscured from therapeutic agents by overlying epithelial cells; or patients who require an implantable device.

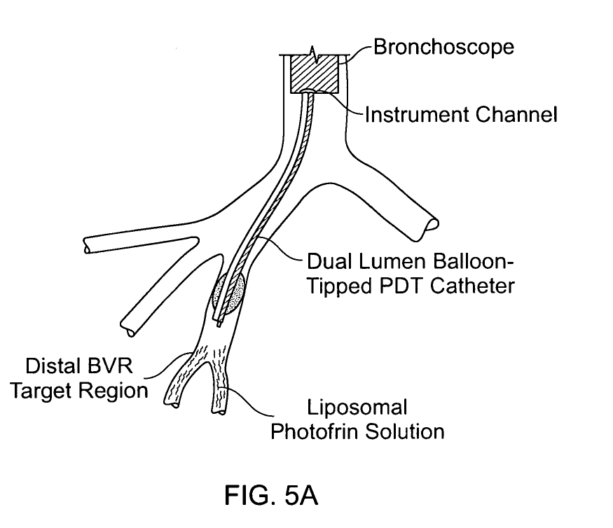

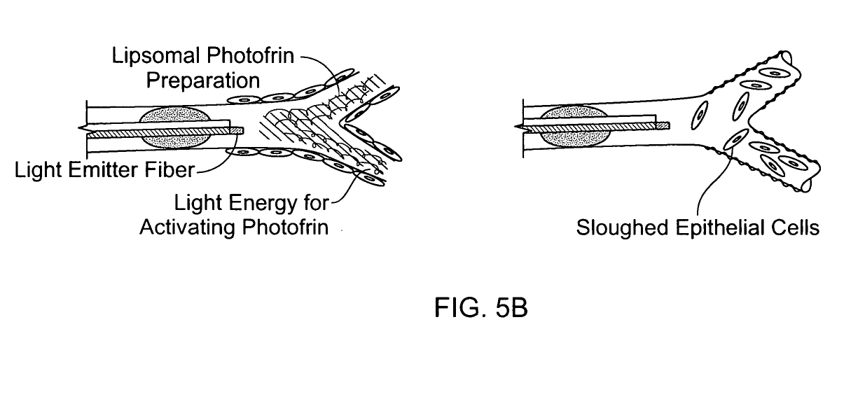

Regardless of the method of delivery, a specialized fiber optic PDT catheter and light wand may be used to administer energy at selected sites. For the purpose of BVR, epithelial “stripping” is necessary at the most distal sites, and thus the catheter system (see FIGS. 5A and 5B) would be designed specifically to ensure application of appropriate light energy at a very distal site. The liposomal photophrin compositions of the present invention may include the phospholipid dipalmitoylphosphatidylcholine (DPPC), a key lipid component of surfactant, which is readily taken up by epithelial cells. Light intensity, wavelength, and generation are selected based on studies conducted to ensure penetration of cytotoxic effect to a level that affects epithelial cells without causing more extensive damage. In a preferred embodiment, an anti-surfactant, suction or mechanical blockage of the airway is then applied to induce regional collapse. As described above, the induction of regional collapse is followed by injection of a reagent (e.g., a fibrin-based hydrogel) to promote scar formation and help secure the area of collapse. Those of ordinary skill in the art may refer to one of the following publications for additional guidance in performing PDT: Kreimer-Birnbaum, Seminars in Hematology 2612:157-173, 1989; Koenig et al., “PDT of Tumor-Bearing Mice Using Liposome Delivered Texaphyrins,” International Conference, Milan, Italy, Biosis citation only, Jun. 24-27, 1992; Berlin et al., Biotechn. Bioengin.: Combin. Chem. 61;107-118, 1998; and Richert, J. Photochem. Photobiol., 19:67-69, 1993.

### Tissue Collapse and Fibrosis

When the target tissue is the lung, any of the conditioning steps described above can be followed by application of a physiologically compatible composition containing an anti-surfactant (i.e., an agent that increases the surface tension of fluids lining the alveoli).

Preferably, the composition is formulated as a solution or suspension and includes fibrin or fibrinogen. An advantage of administering these substances is that they can each act not only as anti-surfactants, but can participate in the adhesive and fibrotic process as well. Optionally, the targeted region can be lavaged with saline to reduce the amount of surfactant that is naturally present prior to administration of the anti-surfactant composition.

Adhesives can be applied to tissue mating surfaces and/or target vessels before the surfaces are brought into contact. The adhesive may be applied to either or both of the mating surfaces and may be a one-part or a two-part adhesive. Further, the curing of the adhesive may be activated by light or heat energy. The adhesive may be applied as a liquid or as a solid film. Preferred adhesive materials include collagen, albumin, fibrin, hydrogel and glutaraldehyde. Other adhesives such as cyano-acrylates may also be used.

### Fibrinogen-based Solutions

Fibrinogen can function as an anti-surfactant because it increases the surface tension of fluids lining the alveoli, and it also can function as a sealant or adhesive because it can participate in a coagulation cascade in which it is converted to a fibrin monomer that is then polymerized and cross-linked to form a stable mesh, permanently stabilizing collapsed regions. Fibrinogen, which has also been called Factor I, represents about 2-4 g/L of blood plasma protein, and is a monomer that consists of three pairs of disulfide-linked polypeptide chains designated (Aα)₂, (Bβ)₂, and γ₂. The “A” and “B” chains represent the two small N-terminal peptides and are also known as fibrinopeptides A and B, respectively. The cleavage of fibrinogen by thrombin results in a compound termed fibrin I, and the subsequent cleavage of fibrinopeptide B results in fibrin II. Although these cleavages reduce the molecular weight of fibrinogen only slightly, they nevertheless expose the polymerization sites. In the process of normal clot formation, the cascade is initiated when fibrinogen is exposed to thrombin, and this process can be replicated in the context of lung volume reduction when fibrinogen is exposed to an activator such as thrombin, or an agonist of the thrombin receptor, in an aqueous solution containing calcium (e.g. 1.5 to 5.0 mM calcium).

The fibrinogen-containing composition can include 3-12% fibrinogen and, preferably, includes approximately 10% fibrinogen.in saline (e.g., 0.9% saline) or another physiologically acceptable aqueous solution. The volume of anti-surfactant administered will vary, depending on the size of the region of the lung, as estimated from review of computed tomagraphy scanning of the chest. For example, the targeted region can be lavaged with 10-100 mls (e.g., 50 mls) of fibrinogen solution (10 mg/ml). To facilitate lung collapse, the target region can be exposed to (e.g., rinsed or lavaged with) an unpolymerized solution of fibrinogen and then exposed to a second fibrinogen solution that is subsequently polymerized with a fibrinogen activator (e.g., thrombin or a thrombin receptor agonist).

The anti-surfactant can contain fibrinogen that was obtained from the patient before the non-surgical lung reduction procedure commenced (i.e., the anti-surfactant or adhesive composition can include autologous fibrinogen). The use of an autologous substance is preferable because it eliminates the risk that the patient will contract some form of hepatitis (e.g., hepatitis B or non A, non B hepatitis), an acquired immune deficiency syndrome (AIDS), or other blood-transmitted infection. These infections are much more likely to be contracted when the fibrinogen component is extracted from pooled human plasma (see, e.g., Silberstein et al., Transfusion 28:319-321, 1988). Human fibrinogen is commercially available through suppliers known to those of skill in the art or may be obtained from blood banks or similar depositories.

Polymerization of fibrinogen-based anti-surfactants can be achieved by adding a fibrinogen activator. These activators are known in the art and include thrombin, batroxobin (such as that from B. Moojeni, B. Maranhao, B. atrox, B. Ancrod, or A. rhodostoma), and thrombin receptor agonists. When combined, fibrinogen and fibrinogen activators react in a manner similar to the final stages of the natural blood clotting process to form a fibrin matrix. More specifically, polymerization can be achieved by addition of thrombin (e.g., 1-10 units of thrombin per ng of fibrinogen). If desired, 1-5% (e.g., 3%) factor XIIIa transglutaminase can be added to promote cross-linking.

In addition, one or more of the compositions applied to achieve lung volume reduction (e.g., the composition containing fibrinogen) can contain a polypeptide growth factor. Numerous factors can be included. Platelet-derived growth factor (PDGF) and those in the fibroblast growth factor and transforming growth factor-β families are preferred. For example, the polypeptide growth factor included in a composition administered to reduce lung volume (e.g., the fibrinogen-, fibrinogen activator-, or fibrin-based compositions described herein) can be basic FGF (bFGF), acidic FGF (aFGF), the hst/Kfgf gene product, FGF-5, FGF-10, or int-2. The nomenclature in the field of polypeptide growth factors is complex, primarily because many factors have been isolated independently by different researchers and, historically, named for the tissue type used as an assay during purification of the factor. This complexity is illustrated by basic FGF, which has been referred to by at least 23 different names (including leukemic growth factor, macrophage growth factor, embryonic kidney-derived angiogenesis factor 2, prostatic growth factor, astroglial growth factor 2, endothelial growth factor, tumor angiogenesis factor, hepatoma growth factor, chondrosarcoma growth factor, cartilage-derived growth factor 1, eye-derived growth factor 1, heparin-binding growth factors class II, myogenic growth factor, human placenta purified factor, uterine-derived growth factor, embryonic carcinoma-derived growth factor, human pituitary growth factor, pituitary-derived chondrocyte growth factor, adipocyte growth factor, prostatic osteoblastic factor, and mammary tumor-derived growth factor). Thus, any factor referred to by one of the aforementioned names is within the scope of the invention.

The compositions can also include “functional polypeptide growth factors,” i.e., growth factors that, despite the presence of a mutation (be it a substitution, deletion, or addition of amino acid residues) retain the ability to promote fibrosis in the context of lung volume reduction. Accordingly, alternate molecular forms of polypeptide growth factors (such as the forms of bFGF having molecular weights of 17.8, 22.5, 23.1, and 24.2 kDa) are within the scope of the invention (the higher molecular weight forms being colinear N-terminal extensions of the 17.8 kDa bFGF (Florkiewicz et al., Proc. Natl. Acad. Sci. USA 86:3978-3981, 1989)).

It is well within the abilities of one of ordinary skill in the art to determine whether a polypeptide growth factor, regardless of mutations that affect its amino acid content or size, substantially retains the ability to promote fibrosis as would the full length, wild type polypeptide growth factor (i.e., whether a mutant polypeptide promotes fibrosis at least 40%, preferably at least 50%, more preferably at least 70%, and most preferably at least 90% as effectively as the corresponding wild type growth factor). For example, one could examine collagen deposition in cultured fibroblasts following exposure to full-length growth factors and mutant growth factors. A mutant growth factor substantially retains the ability to promote fibrosis when it promotes at least 40%, preferably at least 50%, more preferably at least 70%, and most preferably at least 90% as much collagen deposition as does the corresponding, wild-type factor. The amount of collagen deposition can be measured in numerous ways. For example, collagen expression can be determined by an immunoassay. Alternatively, collagen expression can be determined by extracting collagen from fibroblasts (e.g., cultured fibroblasts or those in the vicinity of the reduced lung tissue) and measuring hydroxyproline.

The polypeptide growth factors useful in the invention can be naturally occurring, synthetic, or recombinant molecules and can consist of a hybrid or chimeric polypeptide with one portion, for example, being bFGF or TGFβ, and a second portion being a distinct polypeptide. These factors can be purified from a biological sample, chemically synthesized, or produced recombinantly by standard techniques (see, e.g., Ausubel et al., Current Protocols in Molecular Biology, New York, John Wiley and Sons, 1993; Pouwels et al., Cloning Vectors: A Laboratory Manual, 1985, Supp. 1987).

One of ordinary skill in the art is well able to determine the dosage of a polypeptide growth factor required to promote fibrosis in the context of BLVR. The dosage required can vary and can range from 1-100 nM.

In addition, any of the compositions or solutions described herein for lung volume reduction (e.g., the fibrinogen-based composition described above) can contain one or more antibiotics (e.g., ampicillin, gentamycin, cefotaxim, nebacetin, penicillin, or sisomicin, inter alia). The inclusion of antibiotics in therapeutically applied compositions is well known to those of ordinary skill in the art.

### Fibrin-based Solutions

Fibrin can also function as an anti-surfactant as well as a sealant or adhesive. However, in contrast to fibrinogen, fibrin can be converted to a polymer without the application of an activator (such as thrombin or factor XIIIa). In fact, fibrin I monomers can spontaneously form a fibrin I polymer that acts as a clot, regardless of whether they are crosslinked and regardless of whether fibrin I is further converted to fibrin II polymer. Without limiting the invention to compounds that function by any particular mechanism, it can be noted that when fibrin I monomers come into contact with a patient's blood, the patient's own thrombin and factor XIII may convert the fibrin I polymer to crosslinked fibrin II polymer.

Any form of fibrin monomer that can be converted to a fibrin polymer can be formulated as a solution and used for lung volume reduction. For example, fibrin-based compositions can contain fibrin I monomers, fibrin II monomers, des BB fibrin monomers, or any mixture or combination thereof. Preferably, the fibrin monomers are not crosslinked.

Fibrin can be obtained from any source so long as it is obtained in a form that can be converted to a fibrin polymer (similarly, non-crosslinked fibrin can be obtained from any source so long as it can be converted to crosslinked fibrin). For example, fibrin can be obtained from the blood of a mammal, such as a human, and is preferably obtained from the patient to whom it will later be administered (i.e., the fibrin is autologous fibrin). Alternatively, fibrin can be obtained from cells that, in culture, secrete fibrinogen.

Fibrin-based compositions can be prepared as described in U.S. Pat. No. 5,739,288 (which is hereby incorporated by referenced in its entirety), and can contain fibrin monomers having a concentration of no less than about 10 mg/ml. For example, the fibrin monomers can be present at concentrations of from about 20 mg/ml to about 200 mg/ml; from about 20 mg/ml to about 100 mg/ml; and from about 25 mg/ml to about 50 mg/ml.

The spontaneous conversion of a fibrin monomer to a fibrin polymer can be facilitated by contacting the fibrin monomer with calcium ions (as found, e.g., in calcium chloride, e.g., a 3-30 mM CaCl₂ solution). Except for the first two steps in the intrinsic blood clotting pathway, calcium ions are required to promote the conversion of one coagulation factor to another. Thus, blood will not clot in the absence of calcium ions (but, in a living body, calcium ion concentrations never fall low enough to significantly affect the kinetics of blood clotting; a person would die of muscle tetany before calcium is diminished to that level). Calcium-containing solutions (e.g., sterile 10% CaCl₂) can be readily made or purchased from a commercial supplier.

The fibrin-based compositions described here can also include one or more polypeptide growth factors that promote fibrosis (or scarring) at the site where one region of the collapsed lung adheres to another. Numerous factors can be included and those in the fibroblast growth factor and transforming growth factor-β families are preferred. The polypeptide growth factors suitable for inclusion with fibrin-based compositions include all of those (described above) that are suitable for inclusion with fibrinogen-based compositions.

### Solutions That Include Components of the Extracellular Matrix

The anti-surfactants described above, including fibrin- and fibrinogen-based solutions, can also contain one or more agents that enhance the mechanical and biological properties of the solutions. As described above, such solutions can be used to lavage (i.e. to wash out) the tissue or to adhere one portion of the tissue to another.

Useful agents include those that: (1) promote fibroblast and mononuclear cell chemotaxis and collagen deposition in a self-limited and localized manner; (2) dampen the activity of alveolar epithelial cells, either by inhibiting their ability to express surfactant, which promotes reopening of target regions, or by promoting epithelial cell apoptosis, which causes inflammation; (3) promote epithelial cell constriction, which decreases blood flow to target regions, thereby minimizing mismatching between ventilation and perfusion and any resulting gas exchange abnormalities. More specifically, solutions containing components of the extracellular matrix (ECM), endothelin-1, and/or pro-apoptotic reagents can be. used. Suitable pro-apoptotic agents include proteins in the Bcl-2 family (e.g., Bax, Bid, Bik, Bad, and Bim and biologically active fragments or variants thereof), proteins in the caspase family (e.g., caspase-3, caspase-8, caspase-9, and biologically active fragments or variants thereof), and proteins in the annexin family (e.g. annexin V, or a biologically active fragment or variant thereof). Solutions containing several of these agents have been tested. The first agents to be tested were selected based on their biological attributes, their biophysical effects on gel behavior, their solubility in aqueous solutions (under physiological conditions), and cost. Those of ordinary skill in the art will be able to recognize and use comparable agents without resort to undue experimentation.

The agents selected for use initially were chondroitin sulfate A, low and high molecular weight hyaluronic acid, fibronectin, medium and long chain poly-L-lysine, and the collagen dipeptide proline-hydroxyproline.

Chondroitin sulfate (CS) is an ECM component of the glycosaminoglycan (GAG) family. It is a sulfated carbohydrate polymer composed of repeating dissacharide units of galactosamine linked to glucuronic acid via a beta 1-4 carbon linkage. CS is not found as a free carbohydrate moiety in vivo, but rather is bound to core proteins of various types. As such, it is a component of several important ECM proteoglycans including members of the syndecan family (syndecan 1-4), leucine-rich family (decortin, biglycan), and the hyaluronate binding family (CD44, aggrecan, versican, neuroncan). These CS-containing proteoglycans function in the binding of cell surface integrins and growth factors. CS-containing proteoglycans may function within the lung as scaffolding for collagen deposition by fibroblasts. Thus, ECM components within the glycosaminoglycan family, particularly carbohydrate polymers, are useful in achieving tissue volume reduction (e.g., lung volume reduction carried out bronchoscopically). For example, the addition of chondroitin sulfate A or C at concentrations ranging from 0.05-3.00% has a specific and beneficial effect on both the mechanical and biological properties of fibrin gels. Similarly, solutions useful to lavage and adhere tissue can contain comparable amounts of one or more proteoglycans such as syndecan 1-4, decortin, biglycan, CD44, aggrecan, versican, and neuroncan. In one embodiment, the composition of the invention includes ethanol (e.g., 1-20%) fibrinogen (e.g., 0.01-5.00%), HA (e.g., 0.01-3.00%), FN (e.g., 0.001-0.1%), and CS (e.g., 0.01-1.0%). For example, a useful composition of the invention includes 10% ethanol, 0.5% fibrinogen, 0.3% HA, 0.01% FN, and 0.1% CS.

Hyaluronic acid (HA), like CS, is a polysaccharide, consisting of repeating units of glucuronic acid and N-acetylglucosamine joined by a beta 1-3 linkage. However, unlike CS and other GAGs, HA functions in vivo as a free carbohydrate and is not a component of any proteoglycan family. HA is a large polyanionic molecule that assumes a randomly coiled structure in solution and, because of its self-aggregating properties, imparts high viscosity to aqueous solutions. It supports both cell attachment and proliferation. In addition, HA is believed to promote monocyte/macrophage chemotaxis and to stimulate cytokine and plasmin activator inhibitor secretion from these cells. Thus, polysaccharides that include repeating units of, for example, glucuronic acid and N-acetylglucosamine, are useful in achieving tissue volume reduction (e.g., lung volume reduction carried out bronchoscopically). For example, the addition of either high or low MW HA at concentrations ranging from 0.05-3.00% will have a specific and beneficial effect on both the mechanical and biological properties of fibrin gels.

Fibronectin (Fn) is a widely distributed glycoprotein present within the ECM. It is present within tissues as a heterodimer in which the subunits are covalently linked by a pair of disulfide bonds near the carboxyl terminus. Fn is divided into several domains, each of which has a distinct function. The amino terminal region has binding sites for fibrin, heparin, factor XIIIa, and collagen. Fn has a central cell-binding domain, which is recognized by the cell surface integrins of macrophages, as well as fibroblasts, myofibroblasts, and undifferentiated interstitial cells. Fn's primary function in vivo is as a regulator of wound healing, cell growth, and differentiation. Fn can promote binding and chemotaxis of fibroblasts. It can also act as a cell cycle competency factor allowing fibroblasts to replicate more rapidly when exposed to appropriate “progression signals.” In vitro, Fn promotes fibroblast migration into plasma clots. In addition, Fn promotes alterations in alveolar cell phenotype that result in a decrease in surfactant expression. Thus, Fn molecules that promote tissue collapse and scar formation are useful in achieving tissue volume reduction (e.g., lung volume reduction carried out bronchoscopically). Fn isoforms generated by alternative splicing are useful, and addition of lysophosphatidic acid, or a salt thereof, can be added to Fn-containing solutions to enhance Fn binding. For example, the addition of a Fn at a concentration ranging from 0.05-3.00% will have a specific and beneficial effect on both the mechanical and biological properties of fibrin gels used, for example, in BLVR.

Poly-L-lysine (PLL) is commonly used in cell culture experiments to promote cell attachment to surfaces, and it is strongly positively charged. Despite its large size, it dissolves readily in the presence of anionic polysaccharides, including HA and CS. Thus, PLL, HA, and CS may be used in combination in solutions to lavage, destabilize, and adhere one portion of a tissue to another. The studies described below explore the possibility that PLL in a fibrin network containing long chain polysaccharides generates ionic interactions that make fibrin gels more elastic and less prone to breakage during repeated stretching. PLL can also promote hydration and swelling once matrices are formed. Thus, a particular advantage of using solutions containing PLL for lung volume reduction is that such solutions make it even less likely that the resulting matrices will be dislodged from the airway. PLL having a molecular weight between 3,000 and 10,000 can be used at concentrations of 0.1 to 5.0%.

The di-peptide proline-hydroxyproline (PHP) is common to the sequence of interstitial collagens (type I and type III). Collagen-derived peptides may act as signals for promoting fibroblast in-growth and repair during the wound healing process. The PHP dipeptide, at concentrations ranging from 2.5-10.0 mM, is as effective as type I and type II collagen fragments in promoting fibroblast chemotaxis in vitro. Thus, PHP di-peptides are useful in achieving tissue volume reduction (e.g., lung volume reduction carried out bronchoscopically). For example, the addition of PHP di-peptides at concentrations ranging from 0.05-3.00% will have a specific and beneficial effect on both the mechanical and biological properties of fibrin gels.

The addition of ECM components to washout solutions and fibrin gels may promote tissue collapse and scarring by modulating the activity of interstitial fibroblasts and lung macrophages. Disruption of intact epithelium tends to promote permanent atelectasis and scarring. Thus, it can be useful to expose the alveolar epithelium to agents that cause inflammation and trigger an “ARDS-like” response. Of course, administration of such agents must be carefully controlled and monitored so that the amount of inflammation produced is not hazardous. Alternatively, tissue repair and volume reduction can be facilitated by the addition of agents that promote epithelial cell apoptosis, “programmed cell death,” without extensive necrosis and inflammation. These agents would cause a loss of alveolar cell function without inflammation. One way to produce such a response is by administering sphingomyelin (SGM), a lipid compound that is taken up by certain cell types and enzymatically converted by sphingomyelinase and ceramide kinase to ceramide-1-phosphate, a key modulator of programmed cell death. The application of SGM is also likely to inhibit surfactant, since SGM has anti-surfactant activity in vitro. SGM could be administered in the anti-surfactant washout solution, where it could act specifically on the epithelial surface to destabilize the local surface film and cause epithelial cell death without inflammation. Solutions useful for repairing air leaks in pulmonary tissue or for performing BLVR can contain SGM, or a biologically active variant thereof, at concentrations ranging from 0.05-15.00% (e.g., 0.1, 0.5, 1.0, 2.0, 2.5, 5.0, 7.5, 10.0, 12.0, 13.0, 14.0, or 14.5%).

The efficacy of BLVR can also be enhanced by modulating the endothelial cell response. For example, transient vasoconstriction can be achieved by including epinephrine or norepinephrine in the washout solution. Sustained endothelial modulation could be achieved by inclusion of one of the endothelins, a family of cytokines that promotes vasoconstriction and acts as a profibrotic agent. Endothelin-1, endothelin-2, or endothelin-3 can be used alone or in combination. Thus, solutions of the invention can also include a vasoactive substance such as endothelin, epinephrine, or norepinephrine (at concentrations ranging from 0.01-5.00%), or combinations thereof. The advantage of including one or more vasoactive substances is that they favorably modulate the vascular response in the target tissue and this, in turn, reduces ventilation perfusion mismatching, improves gas exchange, and, simultaneously, promotes scar formation.

Application of fibrin-based, fibrinogen-based, and ECM-containing compositions following lung collapse Following pre-conditioning by one of the methods described above, a targeted region of the lung can be collapsed by exposure to one of the fibrin-based, fibrinogen-based, and ECM-containing compositions described above; in addition, these substances can also be applied to adhere one region of the lung to another and to promote fibrosis when the collapse has been induced by other means. For example, the fibrin-based, fibrinogen-based, and ECM-containing compositions described above can be applied after the lung collapses from blockage of airflow into or out of the targeted region. Such blockage can be readily induced by, for example, inserting a bronchoscope into the trachea of an anesthetized patient, inserting a balloon catheter through the bronchoscope, and inflating the balloon so that little or no air passes into the targeted region of the lung. Collapse of the occluded region after the lung is filled with absorbable gas would occur over approximately 5-15 minutes, depending on the size of the region occluded. Alternatively, a fibrinogen- or fibrin-based solution (e.g. a fibrinogen- or fibrin-based solution that contains a polypeptide growth factor), as well as solutions that contain components of the ECM (such as those described herein), ECM-like agents (such as PLL and PHP), vasoactive substances (i.e., substances that cause vasoconstriction), and pro-apoptotic factors (e.g., proteins in the Bc1-2, caspase, and annexin families) can be applied after the lung is exposed to another type of anti-surfactant (e.g., a non-toxic detergent).

### Identifying and Gaining Access to a Target Region of the Lung

Once a patient is determined to be a candidate for BLVR, the target region of the lung can be identified using radiological studies (e.g., chest X-rays) and computed tomography scans. When the LVR procedure is subsequently performed, the patient is anesthetized and intubated, and can be placed on an absorbable gas (e.g., at least 90% oxygen and up to 100% oxygen) for a specified period of time (e.g., approximately 30 minutes). The region(s) of the lung that were first identified radiologically are then identified bronchoscopically.

Suitable bronchoscopes include those manufactured by Pentax, Olympus, and Fujinon, which allow for visualization of an illuminated field. The physician guides the bronchoscope into the trachea and through the bronchial tree so that the open tip of the bronchoscope is positioned at the entrance to target region (i.e., to the region of the lung that will be reduced in volume). The bronchoscope can-be guided through progressively narrower branches of the bronchial tree to reach various subsegments of either lung. For example, the bronchoscope can be guided to a subsegment within the upper lobe of the patient's left lung.

The balloon catheter may then be guided through the bronchoscope to a target region of the lung. When the catheter is positioned within.the bronchoscope, the balloon is inflated so that material passed through the catheter will be contained in regions of the lung distal to the balloon. This is particularly useful in the methods of the present invention, which include the introduction of liquids into the selected region of the lung.

### Formulations and Use

The compositions of the present invention can be formulated as dry powders, and they may be reconstituted before use. For example, a composition having biophysical characteristics appropriate for treating emphysema can be formulated as a dry powder and reconstituted with water (e.g., sterile, preservative-free water) prior to administration. When possible, and whenever preservatives or anti-microbial agents are omitted, the compositions should be reconstituted using full aseptic technique. When full aseptic technique cannot be ensured, reconstitution should take place immediately before use and any unused suspension should be discarded.

The compositions can be supplied in the form of a kit that, in addition to the compositions, contains, for example, a vial of sterile water or a physiologically acceptable buffer. Optionally, the kit can contain an atomizer system to generate particulate matter (atomizers are presently commercially available) and instructions for use and other printed material describing, for example, possible side effects.

Other methods of administration are suitable, and they include all those presently considered appropriate and effective for photodynamic therapy. A direct and effective method is instillation of the surface film into the lung through the trachea. The compositions can be administered as a liquid solution in water or buffered physiological solutions (e.g., saline), and can be administered over a period of several minutes (e.g., 5-15 (e.g., ten) minutes).

A useful mechanism for delivery of the powder into the lungs of a patient is through a portable inhaler device suitable for dry powder inhalation. Many such devices, typically designed to deliver anti-asthmatic agents (e.g., bronchodilators and steroids) or anti-inflammatory agents into the respiratory system are commercially available. The device can be a dry powder inhaler, which can be designed to protect the powder from moisture and to minimize any risk from occasional large doses. In addition, the device can protect the surface film from light and can provide one or more of the following: a high respirable fraction and high lung deposition in a broad flow rate interval; low deviation of dose and respirable fraction; low retention of powder in the mouthpiece; low adsorption to the inhaler surfaces; flexibility in dose size; and low inhalation resistance. The inhaler can be a singledose inhaler or a multi-dose inhaler.

The compositions, in powder form, can be manufactured in several ways, using conventional techniques. One can, if desired, micronize the active compounds (e.g., one or more of the lipids). One can also use a suitable mill (e.g., ajet mill) to produce primary particles in a size range appropriate for maximal deposition in the lower respiratory tract (i.e., under 10 \hat{1}M). For example, one can dry mix lipids and other components of the surface film (e.g., proteins or peptides) and a carrier (where appropriate) and micronize the substances together. Alternatively, the substances can be micronized separately and then mixed. Where the compounds to be mixed have different physical-properties (e.g., hardness or brittleness), resistance to micronization varies, and each compound may require a different pressure to be broken down to suitable particle sizes

It is also possible to dissolve the components first in a suitable solvent (e.g., sterile water or PBS) to obtain mixing on the molecular level. When this is done, one can adjust the pH value to a desired level. To obtain a powder, the solvent should be removed by a process that allows the components of the surface film to retain their biological activity. Suitable drying methods include vacuum concentration, open drying, spray drying, and freeze-drying. After being dried, the solid material can, if necessary, be ground to obtain a coarse powder, and further, if necessary, micronized.

In addition, and if desired, the micronized powder can be processed to improve the way in which it flows through and out of inhaler (or other) devices. For example, the powder can be processed by dry granulation to form spherical agglomerates with superior handling characteristics. In that case, the device would be configured to ensure that no substantial agglomerates exit the device. A possible advantage of this process is that the particles entering the respiratory tract of the patient are largely within the desired size range.

The delivery apparatus can also be a nebulizer that generates an aerosol cloud containing the components of the surface film. Nebulizers are known in the art and can be a jet nebulizer (air or liquid; see, e.g., EP-A-0627266 and WO 94/07607), an ultrasonic nebulizer, or a pressure mesh nebulizer. Ultrasonic nebulizers, which nebulize a liquid using ultrasonic waves usually developed with an oscillating piezoelectric element, take many forms (see, e.g., U.S. Pat. Nos. 4,533,082 and 5,261,601, and WO 97/29851). Pressure mesh nebulizers, which may or may not include a piezoelectric element, are disclosed in WO 96/13292.

Nebulizers, together with dry powder and metered dose inhalers, are commonly used to deliver substances to the pulmonary air passages. Metered dose inhalers are popular, and they may be used to deliver medicaments in a solubilized form or as a dispersion (the propellant system historically included one or more chlorofluorocarbons, but these are being replaced with environmentally friendly propellants). Typically, these inhalers include a relatively high vapor pressure propellant that forces aerosolized medication into the respiratory tract upon activation of the device. To the contrary, dry powder inhalers generally rely entirely on patients' inspiratory efforts to introduce a medicament in a dry powder form to the lungs. Nebulizers form a medicament aerosol by imparting energy to a liquid solution. More recently, therapeutic agents have been delivered to the lungs during liquid ventilation or pulmonary lavage using a fluorochemical medium.

In a preferred embodiment, the liposomal photophrin compositions of the present invention are delivered to a targeted region of the lung via a bronchoscope.

Although we describe here the detailed methodology for use of a trypsin-based enzymatic pre-conditioning approach, application of any of these alternative epithelial cell preconditioning procedures would be performed in a similar fashion. For example, use of mechanical brushing, ultrasound energy, thermal energy, or photodynamic therapy would each be administered prior to fibrin hydrogel administration. While the specific technique utilized would vary depending upon the approach, the concepts are generally the same and can be expressed as follows: first, remove at least some of the epithelial lining of the target region to facilitate fibroblast proliferation and in-growth; and second, inject the target region with a hydrogel that facilitates attachment, chemotaxis, growth of, and collagen deposition by resident fibroblasts.

The present invention is further illustrated by the following examples, which are provided by way of illustration and should not be construed as limiting. The contents of all references, published patent applications and patents cited throughout the present application are hereby incorporated by reference in their entirety. A number of embodiments of the invention have been described. Nevertheless, it will be understood that various modifications may be made without departing from the spirit and scope of the invention.

### EXAMPLES

### Enzymatic Pre-conditioning

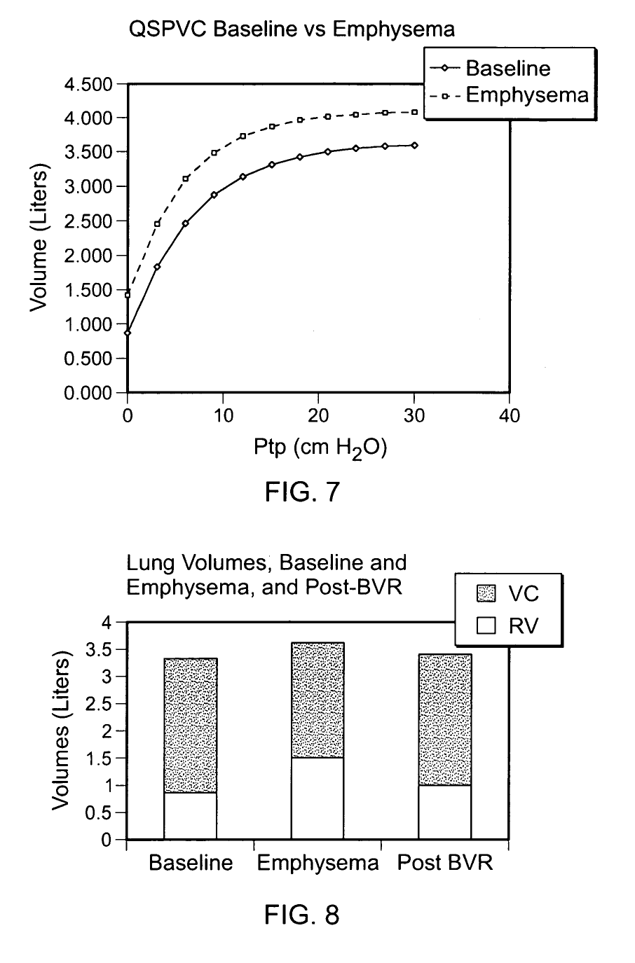

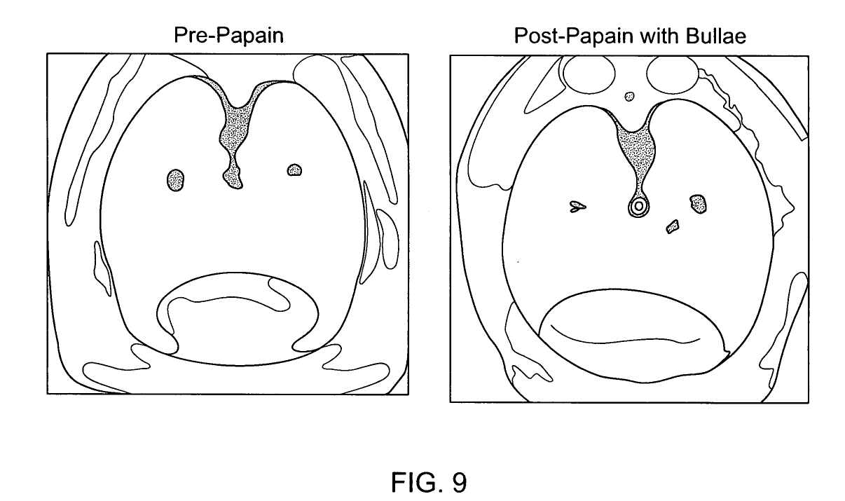

We examined the safety and utility of trypsin pre-conditioning for BVR in a group of seven sheep with emphysema generated by prior exposure to papain inhalation. This large animal model of emphysema is one with which we have extensive prior experience. The model possesses many characteristics of human emphysema, the primary target disease for which BVR has been developed as therapy. In this study, the presence of significant emphysema was demonstrated 2 weeks following serial papain exposure by documenting: (1) a significant increase in lung volumes measured by plethysmography, demonstrating hyperinflation as a result of tissue damage, (2) a significant decrease in tissue density expressed in Houndsfield units as measured by CT scanning; and (3) imaging studies demonstrating readily identifiable regions of bullae formation. The experimental results are summarized in FIGS. 7, 8, and 9.

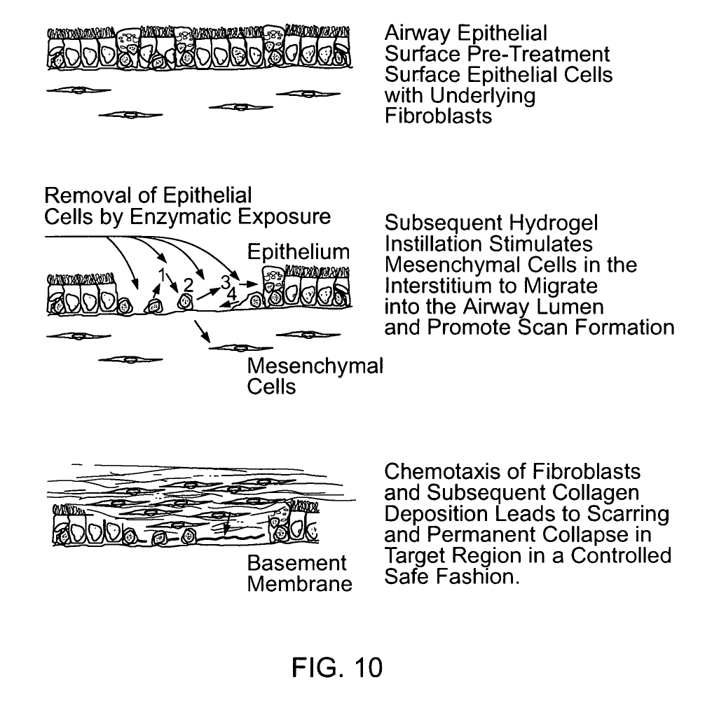

To ensure effective epithelial cell removal, and exposure of the underlying fibroblasts that are the primary cells responsible for scar formation, a critical step in BVR, we employed a trypsin-based solution instilled bronchoscopically into specific targeted regions of lung. The solution requires between 1 and 3 minutes to promote epithelial cell dislodgement. Results presented here were accomplished utilizing a protocol in which the bronchoscope was wedged into position, 15 mls of solution was instilled into a 5^{th}-6^{th} generation airway, and the mixture was left in place for 90 seconds. Suction at −120 cm H₂O was then applied to remove as much of the residual solution as possible. In most instances, returns averaged between 40-50% of instilled volume. A second saline-based washout solution, containing serum and aprotinin, both of which act to neutralize the enzymatic effects of trypsin, was then injected into the same target area. This was left in place for 30 seconds, and suction was then re-applied to remove as much of the mixture as possible. The fibrin based hydrogel was then injected and polymerized within this target area to help maintain a localized reaction, and serve as a substrate for fibroblast attachment and growth as a initial step towards permanent scarring (FIG. 10).

Results: The procedure was uniformly well tolerated by all animals. Trypsin pre-conditioning was associated with no bleeding, excessive coughing, marked hypoxemia, or immunological reactions. Three of seven experienced a mild fever that lasted less than 48 hours. All recovered rapidly from the intervention without the need for immediate or long term oxygen therapy. None required antibiotics, anti-inflammatory agents, or bronchodilator treatment.

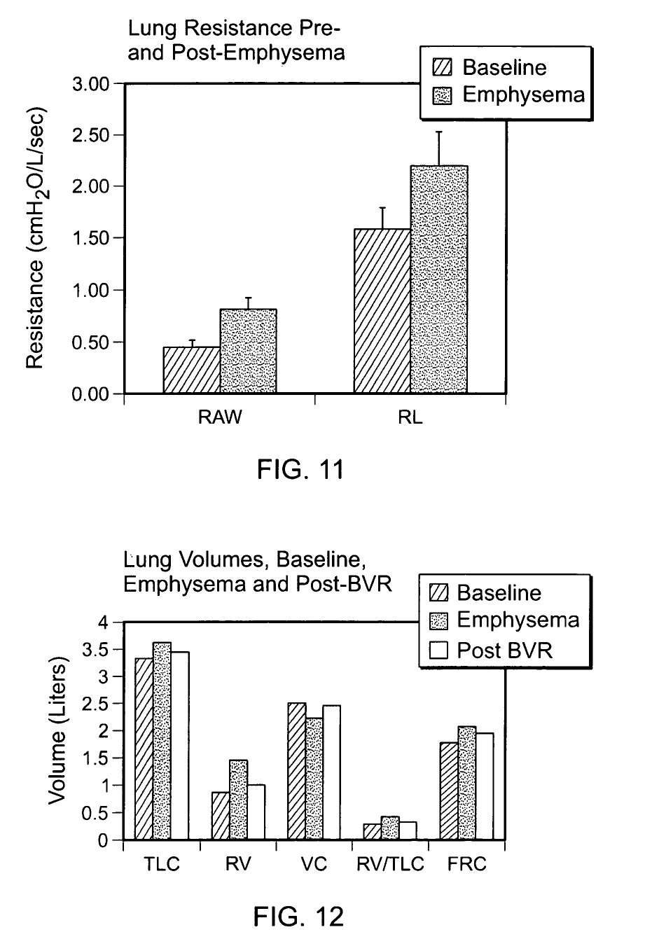

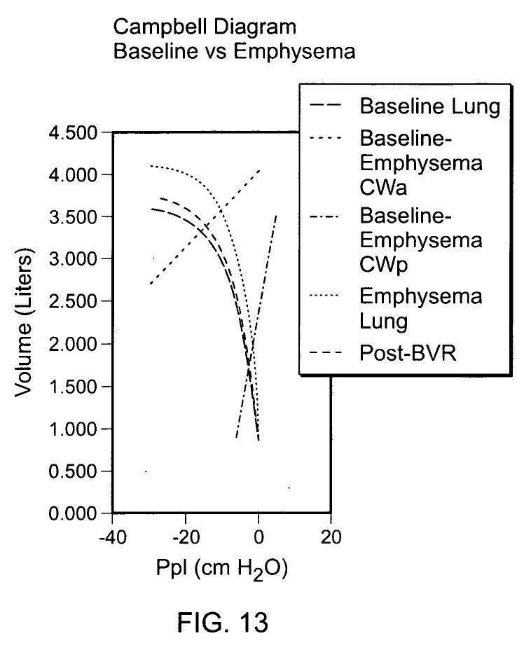

Results of physiology studies for animals undergoing BVR with trypsin pre-conditioning are shown in FIGS. 11, 12 and 13. Compared to baseline, post-papain animals demonstrated a marked increase in airway resistance and lung volumes. At normal respiratory frequencies, total lung resistance (the sum of airway and tissue components) was increased 40±9%, and airway resistance was increased 75±16% (FIG. 11, lung impedance). Total lung capacity (TLC), the total volume within the lung, increased 10±3%, the residual volume (RV), the trapped gas within the lung, decreased 66±21%, and vital capacity (VC), the functional volume within the lung increased 11±4% (FIG. 12, lung volumes including VC). The inter-relationship between chest wall and lung mechanics that ultimately determines the static properties of the respiratory system are summarized in the Campbell diagram (FIG. 13). Emphysema had no Significant impact on either active or passive chest wall mechanics, but caused a significant increase in both TLC and RV. The resulting hyper-inflation caused a decrease in recoil pressures at full inflation from 16.4 cm H₂O to 8.9 cm H₂O.

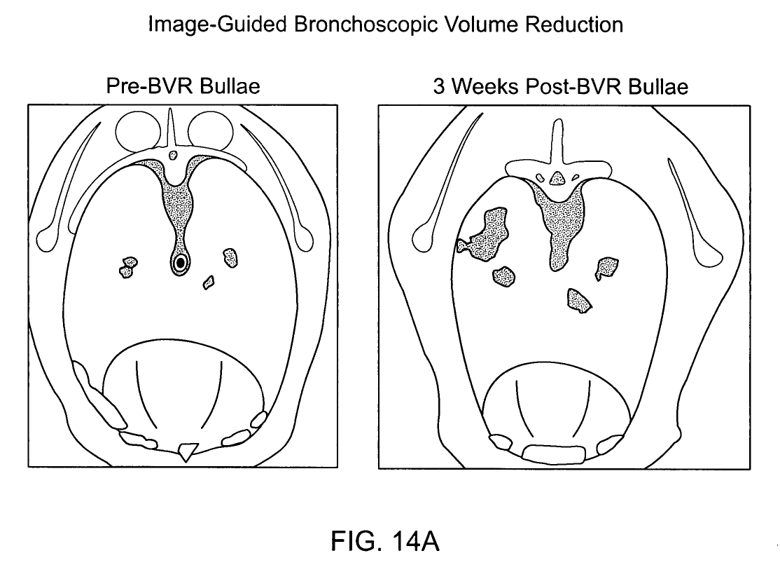

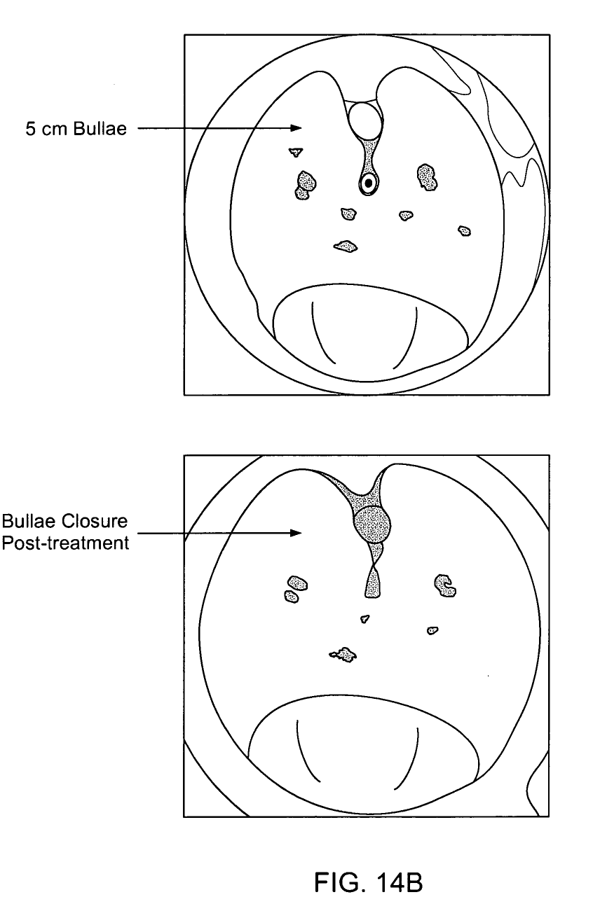

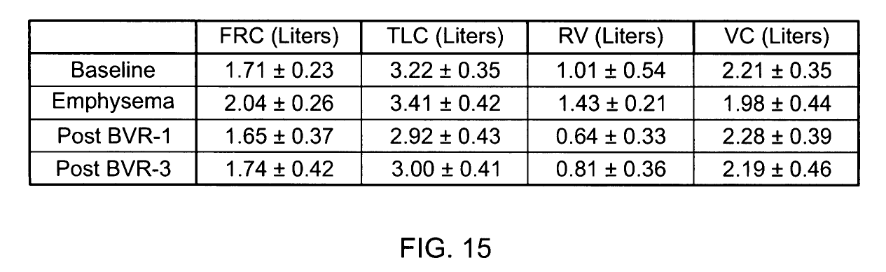

Post BVR studies were performed at 1 and 3 months. The physiological parameters measured are summarized in the table presented as FIG. 15. At both post-treatment time points, a significant reduction in lung volumes was demonstrated. BVR using trypsin pre-conditioning produced significant reductions in TLC (7±2%, p=0.05), RV (30±7%, p=0.01) and RV/TLC (25±6%, p=0.01) ratio with corresponding increases in VC (11±4% p=0.03) and recoil pressures at TLC (69±14%, p=0.007) were decreased. Responses observed at 1 month were sustained at 3 month follow-up demonstrating that BVR treatment using this approach generates what appears to be permanent physiological benefit. FIG. 14 shows an example of an animal with heterogeneous emphysema that had developed a bullous lesion in response to papain instilled bronchoscopically. The bullac located in the right upper dorsal lobe (bronchus R4) measured 5×3×7 cm prior to treatment. At 1 month post BVR, the lesion was reduced in size to 3×2×2 cm in dimensions. At 3 month follow-up, the bullae demonstrated complete closure, with expansion of adjacent normal lung into the region previously occupied by the bullae.

At sites of BVR where poorly localized, homogeneous emphysema had existed, BVR using trypsin pre-conditioning produced localized scars that were readily identified on CT scan, and occurred specifically and exclusively at those sites documented to have undergone BVR injection. Example images of BVR sites treated for presence of diffuse emphysema are also shown in FIG. 14.

At 3-month follow-up, all animals appeared well, were gaining weight, and appeared to have normal activity levels.

### Enzyme Pre-conditioning Solution and Neutralizing Solution

Enzyme pre-conditioning solution: In its preferred formulation, the trypsin pre-conditioning solution consists of an aqueous buffered solution containing 500 BAEE units purified virus free porcine pancreatic trypsin/ml, and 180 mg 4Na-EDTA/ml in pH 7.4 Delbecco's phosphate buffered saline. Although the trypsin source used in this application was porcine, any of multiple sources would be acceptable including human sources and other animal sources.

Trypsin was specifically selected for use here because there is extensive experience utilizing this enzyme in experimentation, it has been shown to have minimal direct cellular toxicity, and is inexpensive to obtain commercially. All of our studies have been performed utilizing trypsin. However, any of several different enzymes with similar characteristics could potentially be utilized for this purpose. Trypsin is a serine protease; multiple enzymes of this class are available commercially, including chymotrypsin, elastase, any of numerous matrix metalloproteinases, or other serine proteases, as disclosed above. Any of these could be used in a formulation for pre-BVR conditioning.

Enzyme “neutralizing” solution: Since each of these enzymes are proteases and have the potential for not only “loosening” epithelial cells as desired but also for damaging underlying tissue structures, we have chosen to neutralize the trypsin washout preparation as an additional safety step during BVR. The results reported above therefore reflect combining trypsin pre-conditioning with neutralization washout.

The neutralizing solution was designed to inactivate serine protease activity and interface well with subsequent instillation of fibrin hydrogel. The composition of the neutralizing solution is as follows: 10% fetal bovine serum; 0.5 mg/ml tetracycline or 1 mg/ml Ciprofloxacin or 1 mg/ml Clindamycin or 0.5 mg/ml Ancef; and 5 mM CaCI₂ dissolved in standard RPMI 1640 cell culture media without glutamine or phenol red, and at pH 7.5.

### Specifics of Method of Application

Prolonged exposure of the lung epithelial surface to trypsin solutions could, in theory, result in tissue damage, and thus a specific protocol for trypsin solution instillation has been developed to limit exposure time. First the bronchoscope is wedged into a specific target region of lung. Given the diameter of the scope for use in human BVR application will be 3-4 mm in diameter, this is likely to correspond to a sub-segmental bronchus. The area subtended by the scope, which corresponds to approximately 5% of total lung volume, is rinsed with 15 mls of enzymatic washout solution. The solution is injected into the target region through the channel of the bronchoscope and left in place for 90 seconds. Then, continuous suction is applied for 1-2 minutes to remove as much of the solution as possible. Thereafter, the neutralizing solution is injected in similar fashion, left in place for 60 seconds, and then suctioned out. The target zone is then ready to be injected with fibrin hydrogel.

A number of embodiments of the invention have been described. Nevertheless, it will be understood that various modifications may be made without departing from the spirit and scope of the invention. Accordingly, other embodiments are within the scope of the following claims.

What is claimed is:

1. A method for performing non-surgical lung volume reduction in a patient, comprising:
(a) administering, through the patient's trachea, a composition comprising an enzyme; and
(b) collapsing a region of the lung, at least a portion of which was contacted by the composition administered in step (a).

2. The method of claim 1, wherein the patient has a chronic obstructive pulmonary disease.

3. The method of claim 2, wherein the patient has emphysema.

4. The method of claim 1, wherein the region of the lung includes an abscess or fistula.

5. The method of claim 1, wherein the enzyme is a protease.

6. The method of claim 5, wherein the protease is a serine protease.

7. The method of claim 6, wherein the serine protease is trypsin, chymotrypsin, elastase, or a matrix metalloproteinase.

8. The method of claim 1, wherein collapsing a region of the lung comprises administering, to the region of the lung, a substance that increases the surface tension of fluids lining the alveoli in the targeted region, the surface tension being increased to the point where the region of the lung collapses.

9. The method of claim 8, wherein the substance is fibrin.

10. The method of claim 9, wherein fibrin is produced in vivo by administration of fibrinogen and a fibrinogen activator.

11. The method of claim 10, wherein the fibrinogen activator is thrombin.
